(19) United States
(12) Patent Application Publication
(10) Pub. No.: US 2022/0018736 A1
(43) Pub. Date: Jan. 20, 2022

(54) DEVICES FOR LATERAL FLOW-BASED BIOLOGICAL SAMPLE COLLECTION AND DIAGNOSIS AND METHODS OF USE THEREOF

(71) Applicant: Bio-Marketing-T, Ltd. (BMT), Or-Akiva (IL)

(72) Inventors: Idan TAMIR, Zichron Yaakov (IL); Nili TAMIR, Zichron Yaakov (IL); Gil PERLBERG, Zichron Yaakov (IL)

(21) Appl. No.: 17/380,879

(22) Filed: Jul. 20, 2021

Related U.S. Application Data
(60) Provisional application No. 63/053,819, filed on Jul. 20, 2020.

(51) Int. Cl. G01N 1/14; G01N 1/20; B01L 3/00

(52) U.S. Cl. CPC G01N 1/14; G01N 1/20; B01L 2300/161; G01N 2001/149; B01L 3/5023

(57) ABSTRACT

A device including a casing first part; a casing second part; a test strip having a sample pad; and a capillary sample collector, wherein the capillary sample collector has an open distal end configured to collect a fluid sample by capillary action and an open proximal end configured to dispense the fluid sample therefrom, wherein, the device is assembled such that the casing first part and the casing second part are joined, wherein a distal end of the casing first part and a distal end of the casing second part are sealed together in a fluid-tight manner, wherein the sample pad is positioned in proximity to the capillary dispensing end, wherein the dispensing end is positioned such that a dispensed fluid sample will be dispensed onto the sample pad, and wherein a dispensing angle between the dispensing and the sample pad of the test strip is less than 10 degrees.

### CROSS-REFERENCE TO RELATED APPLICATION

This is a Section 111(a) application relating to and claiming the benefit of commonly-owned, co-pending U.S. Provisional Patent Application No. 63/053,819, filed on Jul. 20, 2020 and entitled “DEVICES FOR LATERAL FLOW-BASED BIOLOGICAL SAMPLE COLLECTION AND DIAGNOSIS AND METHODS OF USE THEREOF,” the contents of which are incorporated herein by reference in their entirety.

### FIELD OF THE INVENTION

The exemplary embodiments relate to devices for biological sample collection. More particularly, the exemplary embodiments relate to devices for biological sample collection and diagnosis through the use of lateral flow, and related collection and testing methods.

### BACKGROUND

Test strips are used to test biological samples for the presence of biomarkers within the biological samples based on interaction with detector molecules present in the test strips. The sensitivity of such tests can be improved by increasing the amount and concentration of the test sample that reaches the portion of the test strip where the detector molecules are located.

### SUMMARY

In an embodiment, a device includes a casing first part; a casing second part; a test strip having a sample pad; and a capillary sample collector, wherein the capillary sample collector has (a) an open distal end configured to collect a fluid sample by capillary action and (b) an open proximal end configured to dispense the fluid sample therefrom, wherein, the device is assembled such that the casing first part and the casing second part are joined, wherein a distal end of the casing first part and a distal end of the casing second part are sealed together in a fluid-tight manner, wherein the sample pad of the test strip is positioned in proximity to the capillary dispensing end, wherein the dispensing end of the capillary sample collector is positioned such that a dispensed fluid sample will be dispensed onto the sample pad of the test strip, and wherein a dispensing angle between the dispensing end of the capillary sample collector and the sample pad of the test strip is less than 10 degrees.

In an embodiment, the capillary sample collector is segmented into at least two length segments. In an embodiment, a first length segment of the capillary sample collector includes a hydrophilic internal wall, and a second length segment of the capillary sample collector includes a hydrophobic internal wall. In an embodiment, the first length segment of the capillary sample collector is distally positioned so as to allow for fluid sample uptake by capillary forces, and the second length segment of the capillary sample collector is proximally located so as to restrict an amount of fluid collected by the capillary sample collector. In an embodiment, a length of the first length segment of the capillary sample collector is configured to enable collection of a predefined volume of fluid. In an embodiment, the predefined volume is less than 100 microliters. In an embodiment, a first length segment of the capillary sample collector includes a hydrophilic internal wall, and wherein a second length segment of the capillary sample collector includes a hydrophilic internal wall, and wherein the capillary sample collector further comprises an air gap positioned between the first length segment and the second length segment.

In an embodiment, the capillary sample collector has a hydrophilic internal wall, wherein the capillary sample collector forms a 90° angle at the open proximal end, wherein the device includes an air gap between open proximal end of the capillary sample collector and the sample pad of the test strip, and wherein the open proximal end of the capillary sample collector and the air gap cooperate to act as a burst valve at the open proximal end of the capillary sample collector.

In an embodiment, the capillary sample collector is segmented into at least two length segments, wherein a first length segment of the capillary sample collector includes a hydrophilic internal wall, wherein a second length segment of the capillary sample collector includes a hydrophobic internal wall, and wherein a volume of the open proximal end is smaller than a volume of the first length segment of the capillary sample collector, whereby the device is configured not to dilute a sample prior to the sample reaching the sample pad.

In an embodiment, the device is configured such that a portion of a sample fluid dispensed onto the test strip is not diluted. In an embodiment, the portion is less than 50%.

In an embodiment, the capillary sample collector is made of a different material than the casing first part. In an embodiment, the capillary sample collector is made of glass. In an embodiment, the capillary sample collector is made of a polymer. In an embodiment, a plane defined by the capillary sample collector opening is perpendicular to an axis of the capillary sample collector. In an embodiment, a plane defined by the capillary sample collector opening is oriented at an angle of less than 90° to an axis of the capillary sample collector.

In an embodiment, the casing first part includes a groove, and the capillary sample collector is positioned within the groove. In an embodiment, a gap is positioned between the open proximal end of the capillary sample collector and an end of the groove, wherein the gap has a length, and wherein the length of the gap is less than an external diameter of the capillary sample collector. In an embodiment, a casing wall at the end of the groove forms an angle with the test strip sample pad, and wherein the angle is less than 90°. In an embodiment, the angle is less than 60°.

In an embodiment, the device also includes a gasket positioned between the casing first part and the casing second part, wherein the capillary sample collector extends through the gasket.

In an embodiment, the device is configured to collect a fluid sample having a volume that is between 0.5 microliters and 100 microliters.

In an embodiment, the test strip also includes a conjugate pad positioned adjacent the sample pad, wherein a proximal end of the sample pad is positioned above a distal end of the conjugate pad such that the sample pad has a bend angle, and wherein the bend angle is less than 30°.

In an embodiment, fluid collected in the capillary sample tube is visible from outside the device. In an embodiment, diagnostic lines of the test strip are visible from outside the device.

In an embodiment, the casing first part and the casing second part are joined to one another to form a cylindrical casing. In an embodiment, the cylindrical casing is shaped to form an airtight seal when the device is inserted into a tube housing a liquid reagent.

In an embodiment, a method includes providing a capillary sample collector having a distal end and a proximal end, wherein the proximal end of the capillary sample collector has a dispensing opening; collecting a fluid sample at the distal end of the capillary sample collector, wherein the capillary sample collector includes a hydrophilic portion, and wherein the capillary sample collector is configured to limit an amount of the fluid sample; driving a reagent fluid into the distal end of the capillary sample collector, whereby the reagent fluid drives the fluid sample toward the dispensing opening; and dispensing the fluid sample and at least a portion of the reagent fluid through the dispensing opening and onto a test strip sample pad, wherein a flow rate of the sample fluid within the test strip sample pad in a direction toward a conjugate pad is greater than a flow rate of the sample fluid within the test strip sample pad in a direction away from the conjugate pad.

In an embodiment, the capillary sample collector comprises a burst valve between the proximal end and the distal end, and the burst valve is configured to limit the amount of the fluid sample.

In an embodiment, the capillary sample collector further comprises a hydrophobic portion positioned between the hydrophilic portion and the proximal end, and a size of the hydrophilic portion is configured to limit the amount of the fluid sample.

In an embodiment, a dilution of the fluid sample by the reagent fluid is less than 10%.

In an embodiment, the fluid sample is dispensed onto the sample pad at a tilt angle of less than 10°.

In an embodiment, a flow velocity of the fluid sample along the test strip is between 0.1 mm/s and 1 mm/s.

### BRIEF DESCRIPTION OF THE DRAWINGS

FIGS. 1A-1D schematically illustrate operation of an exemplary capillary sample collector.

FIGS. 2A-2E illustrate an exemplary embodiment of a capillary sample collector.

FIG. 3 illustrates a longitudinal cross-section and top view of an exemplary embodiment of a capillary sample collector.

FIGS. 4A-4E illustrate an exemplary embodiment of a capillary sample collector.

FIGS. 5A-5D illustrate embodiments of casing parts of an exemplary embodiment of a capillary sample collector.

FIGS. 6A and 6B illustrate cross-sections of an embodiment of a casing of a capillary sample collector.

FIGS. 7A-7D illustrate exemplary embodiments of a reagent tube and use thereof.

FIGS. 8A and 8B illustrate the operation of an embodiment of a capillary sample collector used in connection with an embodiment of a reagent tube having a reagent absorbed into a porous element.

FIGS. 9A-9F show embodiments of a capillary sample collector and a reagent tube.

FIGS. 10A-10D illustrate operation of an exemplary capillary sample collector and reagent tube.

FIG. 11 shows a flowchart of an exemplary sampling method.

FIG. 12 shows a flowchart of an exemplary sampling method.

### DETAILED DESCRIPTION

The present invention will be further explained with reference to the attached drawings, wherein like structures are referred to by like numerals throughout the several views. The drawings shown are not necessarily to scale, with emphasis instead generally being placed upon illustrating the principles of the present invention. Further, some features may be exaggerated to show details of particular components.

The figures constitute a part of this specification and include illustrative embodiments of the present invention and illustrate various objects and features thereof. Further, the figures are not necessarily to scale, some features may be exaggerated to show details of particular components. In addition, any measurements, specifications and the like shown in the figures are intended to be illustrative, and not restrictive. Therefore, specific structural and functional details disclosed herein are not to be interpreted as limiting, but merely as a representative basis for teaching one skilled in the art to variously employ the present invention.

Among those benefits and improvements that have been disclosed, other objects and advantages of this invention will become apparent from the following description taken in conjunction with the accompanying figures. Detailed embodiments of the present invention are disclosed herein; however, it is to be understood that the disclosed embodiments are merely illustrative of the invention that may be embodied in various forms. In addition, each of the examples given in connection with the various embodiments of the invention which are intended to be illustrative, and not restrictive.

Throughout the specification and claims, the following terms take the meanings explicitly associated herein, unless the context clearly dictates otherwise. The phrases “in one embodiment” and “in some embodiments” as used herein do not necessarily refer to the same embodiment(s), though they may. Furthermore, the phrases “in another embodiment” and “in some other embodiments” as used herein do not necessarily refer to a different embodiment, although they may. Thus, as described below, various embodiments of the invention may be readily combined, without departing from the scope or spirit of the invention.

The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.” Spatial or directional terms, such as “left”, “right”, “inner”, “outer”, “above”, “below”, and the like, are not to be considered as limiting as the invention can assume various alternative orientations. All numbers used in the specification are to be understood as being modified in all instances by the term “about”. The term “about” means a range of plus or minus ten percent of the stated value.

Unless otherwise indicated, all ranges or ratios disclosed herein are to be understood to encompass any and all subranges or sub-ratios subsumed therein. For example, a stated range or ratio of “1 to 10” should be considered to include any and all subranges between (and inclusive of) the minimum value of 1 and the maximum value of 10; that is, all subranges or sub-ratios beginning with a minimum value of 1 or more and ending with a maximum value of 10 or less, such as but not limited to, 1 to 6.1, 3.5 to 7.8, and 5.5 to 10.

The exemplary embodiments relate to testing systems including a capillary sample collector and a test strip integrated into a single casing, along with a reagent tube storing a reagent. In some embodiments, the capillary sample collector is configured to collect a defined volume of a biological sample. In some embodiments, the casing is configured such that, when inserted into the reagent tube, the reagent urges the biological sample out of the capillary sample collector in a manner such that the biological sample travels generally linearly.

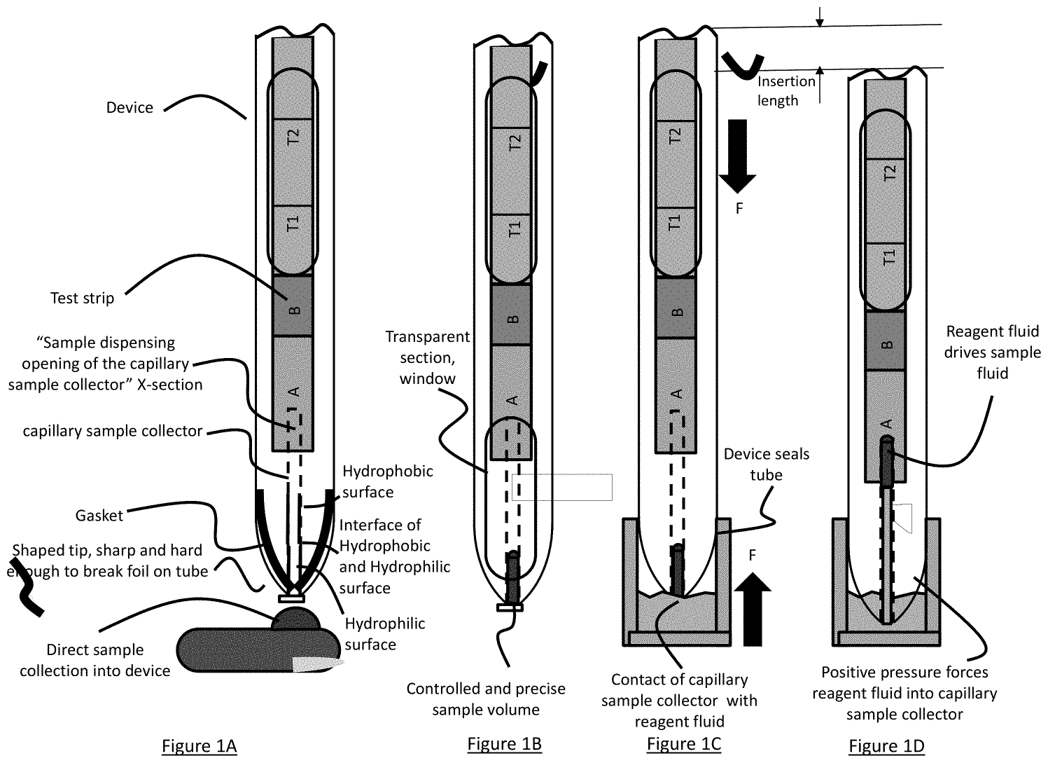

In some embodiments, to collect a predetermined amount of bodily fluid, an exemplary device uses a capillary collector. FIGS. 1A-1D schematically illustrate operation of an exemplary capillary sample collector. In some embodiments, this capillary sample collector is shaped in the form of a tube, with its inner wall coated by a hydrophilic substance to facilitate capillary flow along the length of the tube. In some embodiments, this is accomplished by starting from a hydrophilic building material, such as glass, for the capillary sample collector. In some embodiments, this is accomplished by starting with a hydrophobic building material, such as polystyrene, and coating the surface by a layer of surface-active compound, such as a detergent.

In some embodiments, to collect a predetermined biological sample volume by the capillary sample collector, it is required to prevent the biological fluid from further flowing in the capillary sample collector tube beyond a certain, predetermined point. In some embodiments, to achieve this, a hydrophobic coating is applied to the inner wall of the capillary sample collector beyond the point at which further capillary flow is to be prevented. In some embodiments, such hydrophobic coating is applied to a hydrophilic surface by dipping the section to be coated in a solution of a hydrophobic material, such as a solution of a hydrophobic polymer (e.g., polystyrene) in an organic solvent, or is accomplished by using a hydrophobic surface-rendering reagent such as a alkyltrichlorosilane. In some embodiments, the capillary sample collector is made of two sequential parts, one hydrophilic for sample collection and the other hydrophobic for blocking capillary flow, with their ends being in fluid communication with one another.

In some embodiments, differential coating of specific sections of the capillary sample collector wall by such hydrophilic and hydrophobic substances allows the rate and distance by which a biological fluid sample (e.g., blood) rises through the capillary sample collector to be controlled, further allowing the capture of a predetermined volume of bodily fluid sample within the capillary sample collector, limiting its capacity to such a predetermined bodily fluid volume.

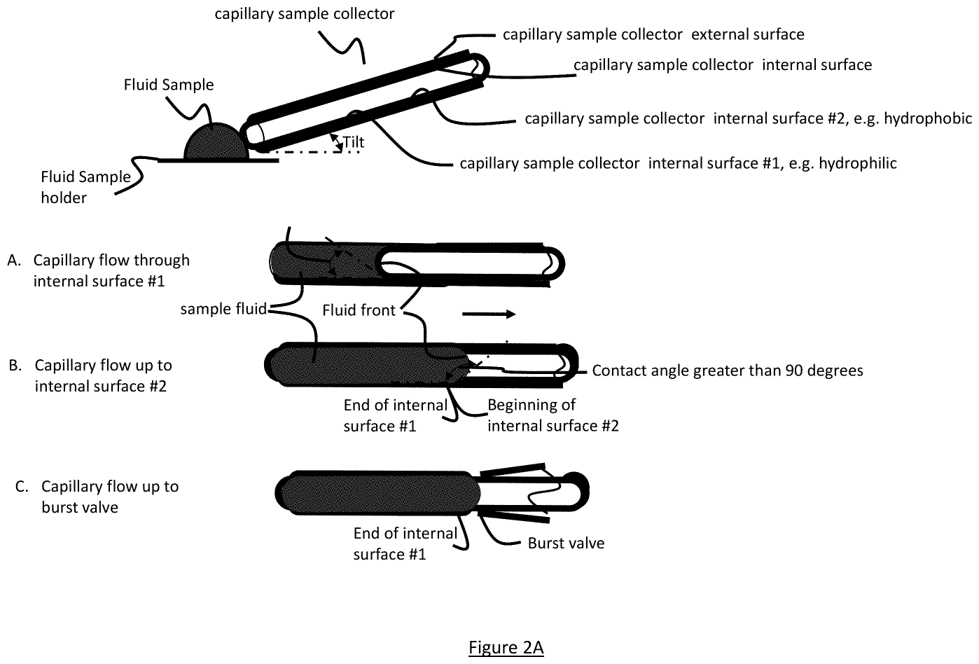

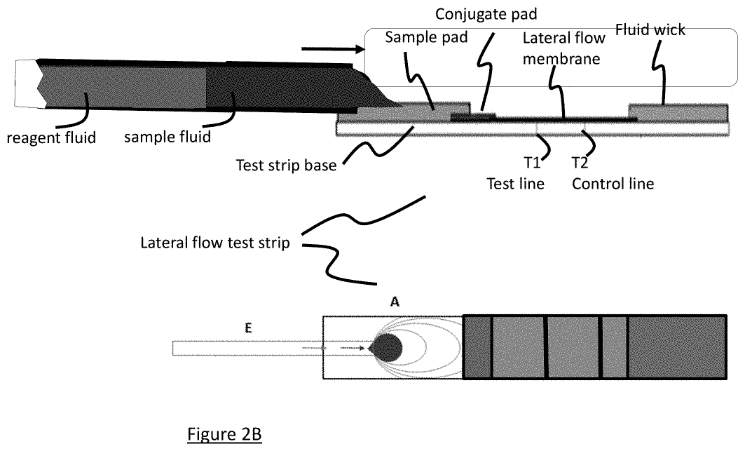

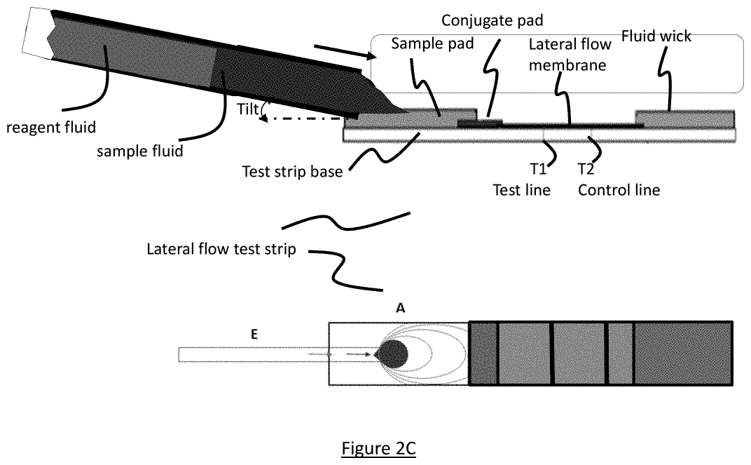

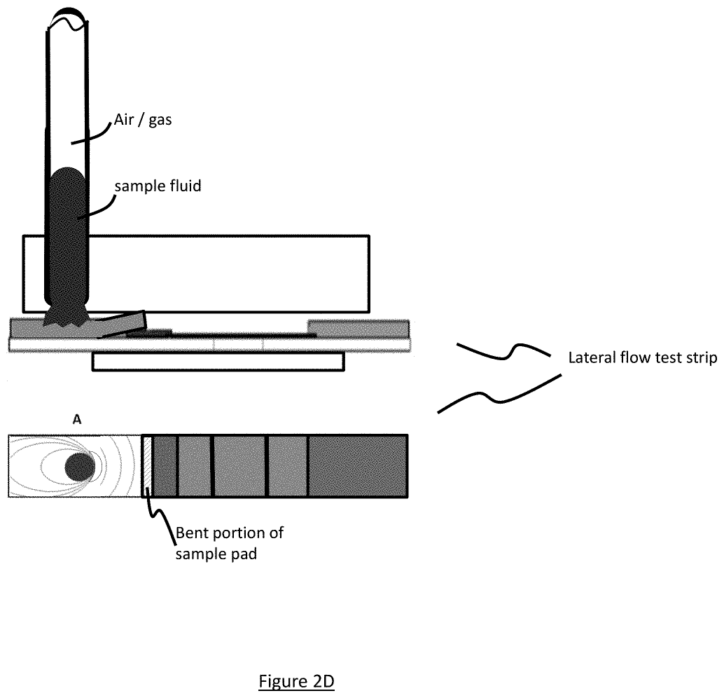

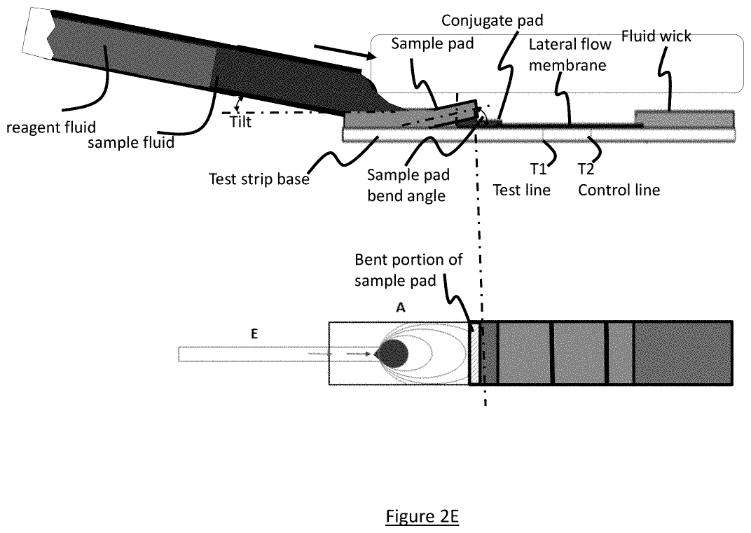

FIG. 2A illustrates an exemplary embodiment of a capillary sample collector. In the embodiment shown in FIG. 2A, a capillary sample collector in tubular form made of a hydrophobic wall material is further segment-wise coated internally by a hydrophilic material or made from such hydrophilic building material, internal surface #1. In some embodiments, when a biological fluid is introduced to the hydrophilic-coated side of the sample collector, the biological fluid flows by capillary forces along the sample collector with a contact angle with the tube's wall that is <90° (A), until it reaches the hydrophobic wall section, internal surface #2, where the contact angle between the fluid and the hydrophobic sample collector wall increases to above 90°, preventing further capillary flow (B).

In some embodiments, a restriction in a capillary tube is used to stop capillary fluid flow. In some embodiments, the opening of the capillary tube beyond such restriction creates a contact angle with the biological fluid that can become larger than 90°, thereby preventing capillary flow beyond such point (also known as a capillary burst valve). In some embodiments, the capillary tube dispensing end is configured as a blunt edge, resulting in an angle of 90° formed between the capillary tube wall and the facet of such dispensing end. As discussed above, in some embodiments, such discontinuity in the capillary wall, as can be seen in FIGS. 2A-C, can serve as a capillary burst valve, preventing additional capillary rise. Additionally, in some embodiments, two capillary tubes are joined with a gap between their ends, creating a wall discontinuity that stops capillary flow. These exemplary embodiments are provided to illustrate different capillary sample collector designs that achieve the purpose of a capillary valve, but it will be apparent to those of skill in the art that other capillary designs and techniques for stopping spontaneous fluid flow in different capillary media are known in the art and the current invention may take advantage of all such designs and techniques.

In some embodiments, the inner wall hydrophilic surface of the capillary sample collector is coated with at least one blocking material to prevent non-specific target analytes from binding to the hydrophilic wall. Such blocking materials are known in the art, and include, among other ingredients, proteins (such as bovine serum albumin) and detergents (such as polysorbate 20). In some embodiments, the hydrophilic surface is covered by an anti-coagulant (e.g., heparin, EDTA, citric acid, etc.) to prevent blood from clotting inside the capillary sample collector.

In some embodiments, the sample collection process includes bringing a capillary sample collector in contact with the fluid sample and holding it in this position while capillary forces drive the fluid up the capillary tube. In some embodiments, the tilt angle of the capillary sample collector can be less than 10°, or less than 30°, or less than 45°, or less than 90°, or less than 135°, or less than 180°.

In some embodiments, once captured in the capillary sample collector, the biological sample fluid will not flow back out of the capillary, unless a surface with a higher energy and/or area is brought in contact with the capillary sample collector at its sample receiving end. In some embodiments, this allows the capture end of the capillary sample to be brought in contact with the reagent in the reagent tube without risking sample loss from the capillary sample collector to the reagent fluid through backflow. Additionally, in some embodiments, due to the relatively small contact area between the sample held in the capillary sample collector and the fluid reagent in the reagent tube, sample dilution by co-diffusion of the sample into the reagent and vice-versa is minimized. In some embodiments, this feature of minimizing the fluid contact area between the sample in the capillary sample collector and the reagent in the reagent tube assures both simple device operation and non-dilutive sample transfer to the test strip sample pad for analysis, as will be further elaborated in the following sections.

In some embodiments, as described herein with reference to FIGS. 2B and 2C, a lateral flow rapid diagnostic test strip includes components laminated in the following order:

1. A sample pad (A) for fluid sample collection. In some embodiments, the sample pad is comprised of cellulose-based material, glass fiber, or other inert materials. In some embodiments, the sample pad has multiple roles including evenly distributing the sample and directing it to the conjugate pad. In some embodiments, the sample pad is impregnated with buffer salts, proteins, surfactants, and other materials to help control the flow rate of the sample and to make it suitable for the interaction with the detection system. In some embodiments, the sample pad includes a porous material and the pores of the sample pad act as a filter to remove redundant or interfering materials and particles, e.g. red blood cells

2. A conjugate pad. In some embodiments, the conjugate pad comprises a membrane to which a labeled detector molecule has been adsorbed. In some embodiments, the conjugate pad contains all the reagents required for optimized interaction between a detector molecule (e.g., antibody) that has been conjugated to the labeling particle's surface, and a target analyte (e.g., an antigen). In some embodiments, the main role of the conjugate pad is to carry the labeled detector molecules and keep them functionally stable until the test is performed. In some embodiments, this is performed by selection of the conjugate buffer. In some embodiments, the conjugate buffer contains carbohydrates (such as sucrose), which serve as a preservative and a re-solubilization agent for the labeled detector molecule. In some embodiments, when the particle-conjugated detector molecules are dried in the presence of sugar, the sugar molecules form a layer around them, stabilizing their biological structures. In some embodiments, when the sample enters the conjugate pad, these sugar molecules rapidly dissolve, carrying the detector molecule complex with target analyte into the fluid stream.

3. A lateral flow membrane. In some embodiments, the lateral flow membrane is made of nitrocellulose. In some embodiments, the lateral flow membrane is printed with a capture molecule at specific location (T1) and the control capture molecule at (T2) along the lateral flow membrane.

4. An absorbent pad or fluid wick. In some embodiments, the absorbent pad or fluid wick is comprised of highly absorbent material, such as cellulose.

In some embodiments, these membranes are sequentially joined, allowing fluid to migrate from the sample pad to the absorbent pad by capillary forces. In some embodiments, a fluid sample containing the target analyte to be diagnosed is spotted on the sample pad, followed by a fluid reagent challenge to the sample pad that allows the analyte to migrate by capillary forces through the conjugate pad containing the detector molecule(s), resulting in binding of the target analyte in the sample by the labeled detector molecule. In some embodiments, the resulting analyte-detector molecule complex migrates via capillary flow along the lateral flow membrane, resulting in binding of the analyte-detector complex at the analyte-specific capture zone. In some embodiments, the capture zone may include multiple analyte-specific test lines (Ti, e.g., T1, T2, etc.), each specific for a different analyte. In some embodiments, binding of a labeled control molecule at the control line of the lateral flow membrane is used to indicate test integrity. In some embodiments, extra fluid reaches the adsorptive fluid wick driving this entire unidirectional fluid flow.

In some embodiments, the formation of a visible colored line in the test region (Ti, e.g., at location T1, location T2, etc.) indicates a positive test result for analyte (i). In some embodiments, the absence of a colored line in the test region suggests a negative test result. In some embodiments, in the control zone of the membrane, an immobilized capture molecule binds labeled control molecules regardless of test specimen composition. In some embodiments, the resulting visible colored band at T2 confirms proper test integrity.

In some embodiments, the dispensing end of the capillary sample collector is positioned in fluid contact and at approximately the same plane with the sample pad of the lateral flow test strip. In some embodiments, such uniplanar placement of the capillary sample collector and the sample pad results in directing the fluid sample entering the sample pad towards the conjugate pad. In some embodiments, the reagent challenge that follows the sample further propagates the sample towards the lateral flow test strip membrane, reducing its “backward” sorption by the sample pad (see FIGS. 2D and 2E). In some embodiments, such “backward” sorption reduction, in turn, minimizes sample loss and analyte dilution in the test strip.

In some embodiments, placement in approximately the same plane includes a small tilt of the dispensing end of the capillary sample collector with respect to the sample pad of the lateral flow test strip, as shown in FIG. 2C. In some embodiments, the tilt angle is less than 1°, or is less than 3°, or is less than 5°, or is less than 10°, or is less than 30°, or is less than 45°. In some embodiments, since the sample pad overlaps to partially cover the test strip's conjugate pad, the sample pad surface curves upward when it overlaps with the conjugate pad (see FIG. 2E), resulting in an upward tilt angle that can be combined with the dispensing end of the capillary sample collector to achieve a tilt angle between the two. In some embodiments, such tilt angle improves sample and reagent directional flow along the test strip.

In some embodiments, the rate of fluid flow from the capillary sample collector into the test strip is controlled by controlling parameters. In some embodiments, the controlling parameters include a reagent tube backpressure, a capillary diameter, a surface energy of the capillary wall, or a combination of the above. In some embodiments, since the reagent tube backpressure may vary due to differences in external air pressure, or due to the volume of air compressed upon introduction of the device casing into the reagent tube, the capillary sample collector is configured to act as the lateral flow limiting rate element. In some embodiments, configuration of the capillary sample collector to act as the lateral flow limiting rate element is achieved by either reducing the capillary diameter or by reducing the capillary wall surface energy (e.g., by employing a more hydrophobic coating), resulting in fluid flow from the capillary end that can be configured to be lower than, equal to, or greater than the maximal capillary flow of the sample along the test strip membrane.

In some embodiments, to reduce the rate of fluid flow, the proximal end of the capillary sample collector is connected to a hollow needle having an internal diameter that is smaller than the internal diameter of the capillary sample collector. In some embodiments, the hollow needle has a first end (e.g., a receiving end) that is connected to the proximal end of the capillary sample collector and an opposite second end that is positioned so as to be in contact with the sample pad of the test strip. In some embodiments, when a reagent is driven into the distal end of the capillary sample collector, the fluid sample is driven through the hollow needle and on to the sample pad of the test strip.

It will be known to those of skill in the art that reducing lateral flow speed along the lateral flow membrane improves test sensitivity, due to increased interaction time for the detector-analyte complex with the capture molecule at the capture site(s) on the lateral flow membrane. The exemplary embodiments allow controlling the membrane lateral flow speed not only by optimizing membrane porosity to control fluid flow rate along the lateral flow membrane, but also by the controlling the rate of sample and reagent flow through the capillary sample collector and into the sample pad.

FIGS. 2D and 2E show a “sample pad bend angle”. In some embodiments, the sample pad bend angle results from layering the proximal side of the sample pad on top of the conjugate pad. In some embodiments, the sample pad bend angle is defined as the angle between (1) the plane in center of the sample pad above the distal end of the conjugate pad and (2) the plane of the test strip base.

In some embodiments, in the case of a lateral capillary to sample pad configuration, the sample fluid is directed towards the conjugate pad, such that it can overcome the gravitational flow restriction created by the bent portion of the sample pad. In some embodiments, the flow pattern of the sample fluid in the sample pad is directed towards the conjugate pad. As a result, in such embodiments, most of the sample fluid will reach the conjugate pad. In some embodiments, at least 35% of the sample fluid reaches the conjugate pad. In some embodiments, at least 40% of the sample fluid reaches the conjugate pad. In some embodiments, at least 45% of the sample fluid reaches the conjugate pad. In some embodiments, at least 50% of the sample fluid reaches the conjugate pad.

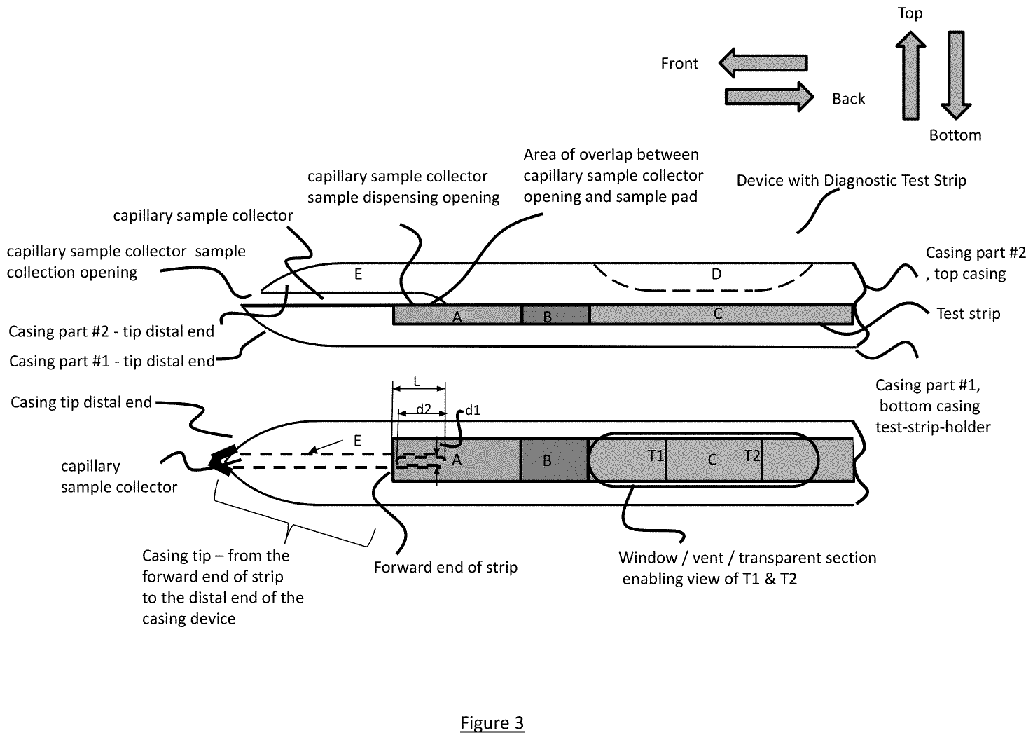

In some embodiments, a casing device for a lateral flow test strip that also serves for collecting a biological fluid sample and transferring it to a diagnostic test strip for diagnosis is as shown in FIG. 3. FIG. 3 shows a longitudinal cross section and a top view of a casing device with a capillary sample collector and a diagnostic test strip included.

In some embodiments, the casing device is configured to house a test strip, to collect a fluid sample, to form a seal with a tube containing a reagent fluid, to drive the sample fluid by the reagent fluid under pressure to the test strip, and to provide a visible indication of diagnostic test results. In some embodiments, the casing device includes two parts. Casing part #1, also referred to as the bottom casing, houses the diagnostic test strip. Casing part #2, also referred to as the top casing, includes a section that enables viewing a portion of the test strip.

In some embodiments, the casing device front distal end includes a capillary opening for sample collection. In some embodiments, the proximal sample dispensing end of the capillary sample collector is fluidly connected to the sample pad of the test strip.

In some embodiments, the capillary sample collector includes a sample dispensing opening that is positioned in proximity to the test strip sample pad A. In some embodiments, in a top view, the capillary sample collector overlays the area of sample pad A. In some embodiments, the capillary sample collector sample dispensing opening is positioned above sample pad A, at a distance L (e.g., from 0 mm to 10 mm) from sample pad A front end distal edge. In other embodiments, the capillary sample collector sample dispensing opening is positioned at a distance L (from (−)10 to 0 mm) from the sample pad A front end distal edge. In such embodiments, the sample fluid that arrives at the dispensing end of the capillary sample collector flows the rest of the distance towards the sample pad of the test strip within a casing groove. In some embodiments, the surfaces of the casing parts are hydrophobic, forming “hydrophobic confinement” for the fluid flowing in the groove. In some embodiments, the hydrophobic confinement prevents fluid flow through the gap between the two parts of the casing.

In some embodiments, the capillary sample collector sample dispensing opening is approximately a blunt (e.g., 90°) ended cylindrically shaped tube, wherein d1 is the smaller dimension of the rectangular opening and d2 is the larger dimension of the rectangular opening. In some embodiments, the capillary sample collector sample dispensing opening is approximately oval shaped, wherein d1 is the smaller dimension of the oval opening and d2 is the larger dimension of the oval opening. In some embodiments, the area of overlap between the capillary sample collector sample dispensing opening and sample pad A is equal to the area of the sample dispensing oval opening.

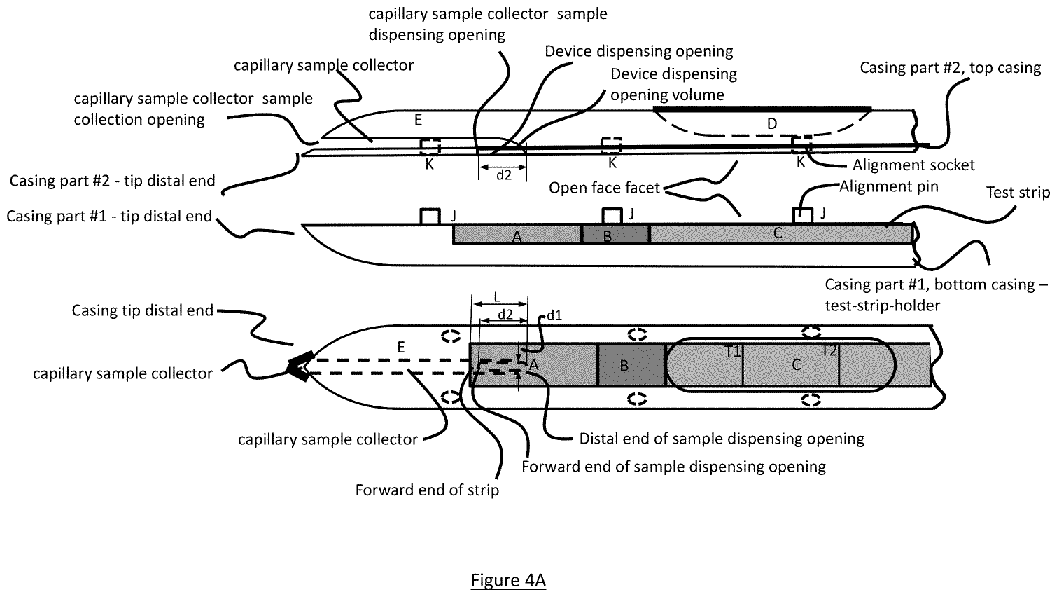

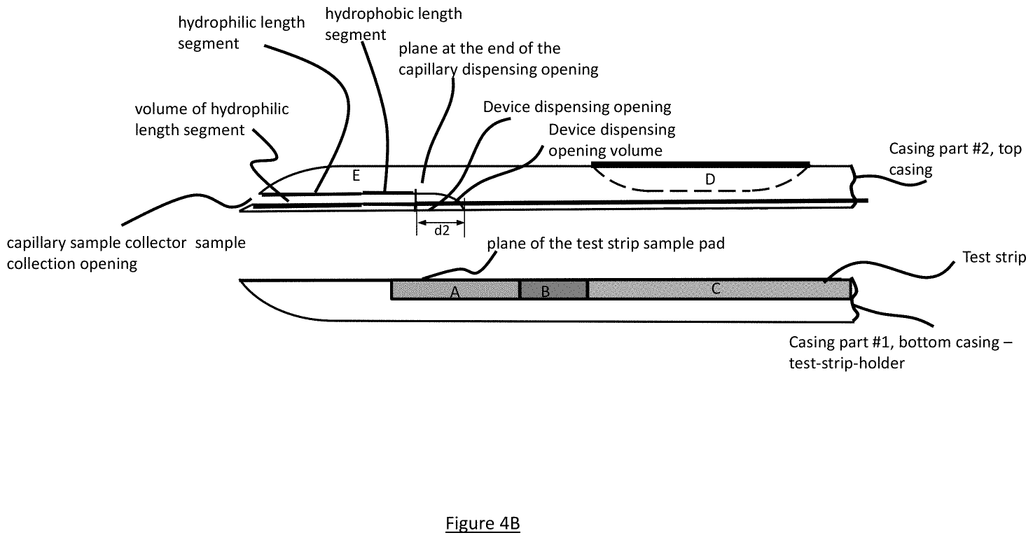

In some embodiments, as shown in FIG. 4A, a device includes a “device dispensing opening” having a “Device dispensing opening volume.” In some embodiments, the capillary sample collector dispenses fluid onto the test strip sample pad through a device dispensing opening. In some embodiments, the volume of the device dispensing opening is bounded by the plane at the end of the capillary dispensing opening, the plane of the test strip sample pad, and the casing part, when the device is assembled.

In some embodiments, the volume of the device dispensing opening is smaller than the volume in the hydrophilic length segment of the capillary sample collector. In some embodiments, the volume of the device dispensing opening is smaller than the volume in the hydrophilic length segment of the capillary sample collector such that a portion of the sample fluid in the hydrophilic length segment will be absorbed by the test strip sample pad before reagent fluid reaches the volume of the device dispensing opening.

In some embodiments, the capillary dispensing opening is adjacent to the test strip sample pad such that fluid being dispensed is directly absorbed by the test strip sample pad.

FIG. 4B shows the volume of the device dispensing opening, as well as the hydrophilic and hydrophobic segments; the hydrophilic volume; the plane at the end of the capillary dispensing opening; the device dispensing opening, through which fluid is dispensed onto the tests strip sample pad; the volume of the device dispensing opening, which is bounded by the plane at the end of the capillary dispensing opening, the plane of the test strip sample pad and the casing part, when the device is assembled; and the plane of the test strip sample pad.

In some embodiments, the section that enables viewing of a portion of the diagnostic test strip is made from a transparent material. In some embodiments, the section that enables viewing a portion of the diagnostic test strip is an opening. In some embodiments, the section that enables viewing a portion of the diagnostic test strip includes a vent that enables a flow of gas (e.g., air).

In some embodiments, the portion of the test strip that is viewable includes a portion of the lateral flow membrane C. In some embodiments, the portion of the test strip that is viewable includes colored lines Ti.

In some embodiments, the top casing part includes a capillary sample collector. In some embodiments, the capillary sample collector is an integral part of the top casing part. In some embodiments, the capillary sample collector is attached to the top casing part. In some embodiments, the capillary sample collector and the top casing part comprise different materials.

In various embodiments, the capillary sample collector may have different geometrical shapes and cross-sections, including, open tubular, porous, or any shape that allows liquid flow by capillary forces. In some embodiments, the capillary sample collector can be used to collect samples of bodily fluids, including whole blood, serum, plasma, or saliva, via capillary forces.

In some embodiments, the capillary sample collector dimensions, length and diameter, depend on the amount of bodily fluid to be collected, which is further dependent on both the concentration of the analyte in the bodily fluid sample and test sensitivity towards that analyte. For example, collecting 25 μL of whole blood (around one drop) can be achieved with a 1 mm diameter round tube having a length of >32 mm.

In some embodiments, the capillary sample collector is formed by integrating at least two parts of the casing device. In some embodiments, the capillary sample collector is formed by the integration of the casing top and bottom parts. In some embodiments, the capillary sample collector is formed by the integration of the casing top and bottom parts and additional parts including a seal, a gasket, an adhesive, and a third part that is integrated within the device.

In some embodiments, the device front distal end, referred to as the casing tip, spans the length from the casings most distal front end to the forward end of the test strip. In some embodiments, the device front distal end includes the distal end of casing part #1 and distal end of casing part #2. In some embodiments, the device front distal end is shaped such that a seal can form with a reagent test tube when the two are connected. In some embodiments, the device front end design and material choice are designed such the device front end can penetrate a foil-covered tube.

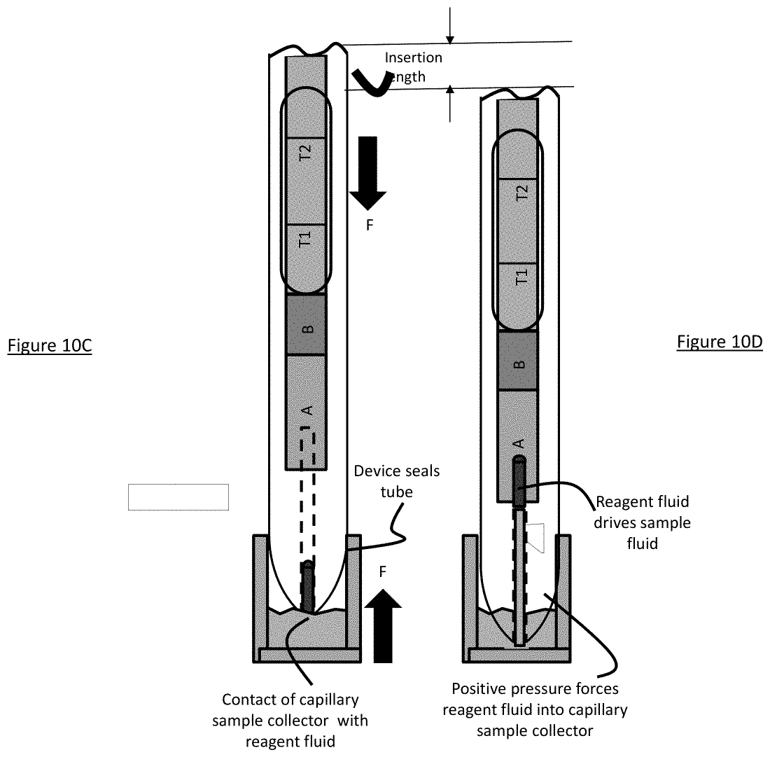

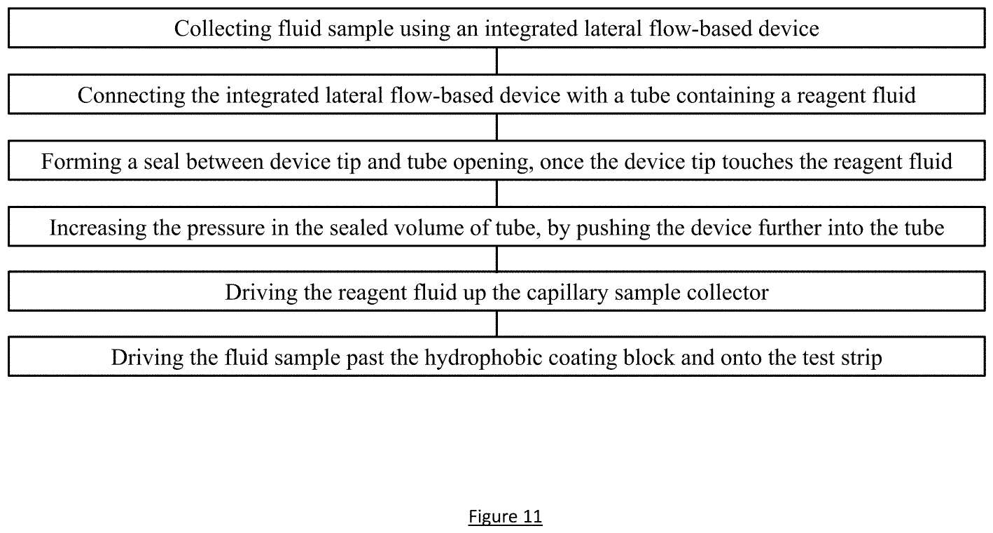

In some embodiments, a portion of the casing tip is transparent such that fluid in the capillary sample collector is visible. In some embodiments, a portion of the casing tip is transparent and includes ruler-like hatch marks (see FIG. 10B). In some embodiments, a portion of the casing tip is transparent, ensuring collection of the correct volume of fluid in the capillary sample collector. In some embodiments, a portion of the casing tip is transparent and enables estimating the volume of fluid in the capillary sample collector.

Referring back to FIG. 4A, a longitudinal cross section and a top view of a casing device parts, with a diagnostic test strip, are shown. In some embodiments, the casing device parts include alignment pins and sockets. In some embodiments, one of the casing parts includes at least one pin while another part includes at least one socket configured to mate with the pin. In some embodiments, one of the casing parts includes multiple pins while the second part includes multiple sockets configured to mate with the pins of the first part. In some embodiments, the pin functions as a molded stud that is press fit into the socket and provides the function of a mechanical fastener. In some embodiments, the casing device parts include sockets in which pins, such as dowel pins, are fitted and pressed into. In some embodiments the pin-in-slot assembly includes a heat source. In some embodiments, the casing device parts are joined by ultrasonic welding.

In some embodiments, the casing parts include snap joints that provide for a snap fit assembly. In some embodiments, one casing part includes a slot whilst the second casing part includes a corresponding wedge, whereby the parts can be joined by sliding the wedge of the second casing parts into the slot of the first casing part.

In some embodiments, the casing parts are assembled with an adhesive. In some embodiments, the casing parts are assembled with an adhesive that is applied to all or substantially all of the contact area between the casing parts. In some embodiments, the casing parts are assembled with an adhesive that is applied to a portion of the contact area between the casing parts. In some embodiments, the adhesive is configured to retain the casing parts in assembled relationship with respect to one another. In some embodiments, the adhesive is configured to provide a fluid-tight seal between the casing parts. In some embodiments, a conformable sealing element (e.g., a gasket) is positioned along at least a portion of the contact area between the casing parts, and cooperates with the adhesive to provide a fluid-tight seal between the casing parts.

In some embodiments, one of the casing device parts includes screw threads. In some embodiments, one of the casing parts includes sockets in which threaded parts can be inserted into.

In some embodiments, assembly of the casing device parts includes multiple assembly methods. In some embodiments, the assembly of the casing device parts includes multiple assembly methods such as alignment pins in slots and an adhesive, alignment pins in slots and a heat source, a sliding slot and wedge and an adhesive, a sliding slot and wedge and a snap joint.

FIG. 4C shows a longitudinal cross-section of a casing device with a diagnostic test strip and an external capillary sample collector. In some embodiments, the capillary sample collector of the test strip casing device is an external capillary sample collector. In some embodiments, the external capillary sample collector includes at least one component that is not an integrated part of the casing parts.

In some embodiments, the external capillary sample collector is a capillary tube made of glass. In some embodiments, the external capillary sample collector is a capillary tube made of a polymeric material.

In some embodiments, the external capillary sample collector is of uniform diameter. In some embodiments, the external capillary sample collector is a straight tube (e.g., is cylindrical). In some embodiments, at least one section of the external capillary sample collector is curved.

In some embodiments, the plane defined by an external capillary sample collector opening is at an angle α to the capillary sample collector axis, as seen in Inset 3 of FIG. 4C. In some embodiments, a is more than 30°, or is more than 40°, or is more than 45°, or is more than 60°, or is more than 75°, or is more than 80°. In some embodiments, the plane defined by an external capillary sample collector opening is perpendicular to the capillary sample collector axis and a equals 90°.

In some embodiments, the external capillary sample collector includes a sample dispensing opening. In some embodiments, the external capillary sample collector sample dispensing end is the capillary sample collector sample dispensing opening.

In some embodiments, the external capillary sample collector sample dispensing opening is a slot in the surface of the tube. In some embodiments, as seen in Inset 1 of FIG. 4C, the external capillary sample collector sample dispensing opening is an enclosed slot in the surface of the tube in proximity to the capillary sample collector sample dispensing end. In some embodiments, as seen in Inset 2 of FIG. 4C, the external capillary sample collector sample dispensing opening is an open-ended slot in the surface of the tube, wherein the open end of the slot joins the opening at the dispensing end of the capillary sample collector.

In some embodiments, at least one of the device casing parts includes a groove in which the external capillary sample collector is positioned. In some embodiments, two of the device casing parts combine to form a groove in which the external capillary sample collector is positioned after assembly of the test strip casing device.

In some embodiments, the external capillary sample collector is positioned such that the sample dispensing opening is in proximity to the test strip sample pad A. In some embodiments, the external capillary sample collector is positioned such that the sample dispensing opening is above the test strip sample pad A. In some embodiments, the external capillary sample collector is positioned such that the sample dispensing opening is above and faces the test strip sample pad A.

In some embodiments, the external capillary sample collector is positioned such that the sample dispensing opening is in contact with the test strip sample pad A. In some embodiments, the external capillary sample collector is positioned such that all of the area of the sample dispensing opening is above the test strip sample pad A. In some embodiments, the external capillary sample collector is positioned such that the sample dispensing opening distal end is at a distance L from the forward end of the test strip. In some embodiments, L is less than 1.0 mm, or is less than 2.0 mm, or is less than 3.0 mm, or is less than 5.0 mm, or is less than 7.0 mm, or is less than 10.0 mm, or is less than 15.0 mm.

In some embodiments, the external capillary sample collector is positioned such that the sample dispensing opening provides fluidic connectivity between the capillary sample collector sample collection opening and the test strip sample pad A.

FIG. 4D shows a longitudinal cross section of casing device parts with a diagnostic test strip and an external capillary sample collector. In some embodiments, the capillary sample collector of the test strip casing device is an external capillary sample collector. In some embodiments, the external capillary sample collector includes at least one component that is not an integrated part of the casing device parts. In some embodiments, casing device #1 (e.g., the top casing half), includes a niche or a groove in which the external capillary sample collector is positioned.

In some embodiments, as shown in Inset 1 of FIG. 4D, an exemplary device includes a gap, having a width e, between the end of the niche and the end of the dispensing opening of the external capillary sample collector. In some embodiments, the width e is a multiple N of the diameter of the external capillary sample collector. In some embodiments, N is less than 0.5, or is less than 0.75, or is less than 1.0, or is less than 1.5, or is less than 2.0, or is less than 2.5, or is less than 3.0, or is less than 4.0, or is less than 5.0.

In some embodiments, as shown in Inset 2 of FIG. 4D, the depth of the niche is defined as De, wherein the depth of the niche is measured from the root of the niche to its upper lip, along the capillary sample collector cross section. In some embodiments, the depth of the niche De is a multiple M of the diameter of the external capillary sample collector. In some embodiments, M is less than 0.1, or is less than 0.25, or is less than 0.5, or is less than 0.75, or is less than 1.0, or is less than 1.5, or is less than 2.0, or is less than 3.0. In one example, as shown in Inset 1 of FIG. 4D, M=1 and the niche depth De1 is equal to the diameter of the external capillary. In another example, as shown in Inset 2 of FIG. 4D, M=0.5 and the niche depth De2 is equal to half the diameter of the external capillary.

In some embodiments, the wall at the end of the niche, also referred to as the “niche end,” is angled. In some embodiments, the angle e-α, shown in Inset 2 of FIG. 4D, is more than 30°, or is more than 40°, or is more than 45°, or is more than 60°, or is more than 75°, or is more than 80°. In some embodiments, the angle e-α is approximately 90°.

In some embodiments, the angled wall directs the flow of the sample fluid exiting the dispensing opening of the external capillary sample collector onto sample pad A. In some embodiments, the magnitude of the angle e-α is a parameter that is configured to direct the flow of the sample fluid exiting the dispensing opening of the external capillary sample collector, on to sample pad A. In some embodiments, the magnitude of the angle e-α is a parameter configured to reduce turbulence in the flow of fluid exiting the dispensing opening of the external capillary sample collector. In some embodiments, a lower angle e-α can reduce turbulence in the flow of fluid exiting the dispensing opening of the external capillary sample collector. For example, an angle e-α of 45° will reduce the turbulence in the flow of fluid exiting the dispensing opening of the external capillary sample collector compared to an angle e-α of 90°.

In some embodiments, the wall at the end of the niche, also referred to as the “niche end,” is concave, such that it forms a cup-like shape also referred to as the “niche end cup.” In some embodiments, the depth of the concavity is p as shown in Inset 3 of FIG. 4D. In some embodiments, the depth p is a multiple Q of the diameter of the external capillary sample collector. In some embodiments, Q is less than 0.1, or is less than 0.2, or is less than 0.3, or is less than 0.4, or is less than 0.5, or is less than 0.75, or is less than 1.0, or is less than 1.5, or is less than 2.0, or is less than 2.5, or is less than 3.0, or is less than 4.0, or is less than 5.0.

In some embodiments, a portion of the sample fluid that is dispensed from the dispensing opening of a capillary sample collector fills the “niche end cup” while the rest is dispensed on sample pad A. In some embodiments, the portion of the sample fluid that is dispensed from the dispensing opening of a capillary sample collector and fills the “niche end cup” is the initial portion of the collected sample fluid. In some cases, the first drop of sample fluid does not contain a representative sample of analytes. In some embodiments, by filling the niche end cup with the first drop of sample fluid, the first drop is not transferred to the sample pad, and the lateral flow test begins with a portion of the sample fluid that is behind the first drop, thereby improving the accuracy of the test results.

FIG. 4E shows a casing part that supports the test strip and the capillary sample collector, as well as the niche in the casing part, from the casing part distal tip to the distal aspect of the sample pad. As shown in FIG. 4E, the niche includes a niche root and a niche lip.

In some embodiments, a capillary sample collector is positioned in a niche in the casing parts, and a portion of the capillary sample collector is positioned on top of the test strip sample pad.

In some embodiments, a capillary sample collector is positioned in a niche in the casing parts, and a portion of the capillary sample collector is positioned on top of the test strip sample pad, forming a depression in the test strip sample pad when the device is assembled.

In some embodiments, a capillary sample collector is positioned in a niche in the casing parts, and the proximal end of the capillary sample collector is positioned adjacent to the distal aspect of the test strip sample pad.

In some embodiments, the niche depth De is smaller than the diameter of the capillary sample collector. In some embodiments, the niche depth De is approximately the diameter of the capillary sample collector. In some embodiments, the niche depth De is larger than the diameter of the capillary sample collector.

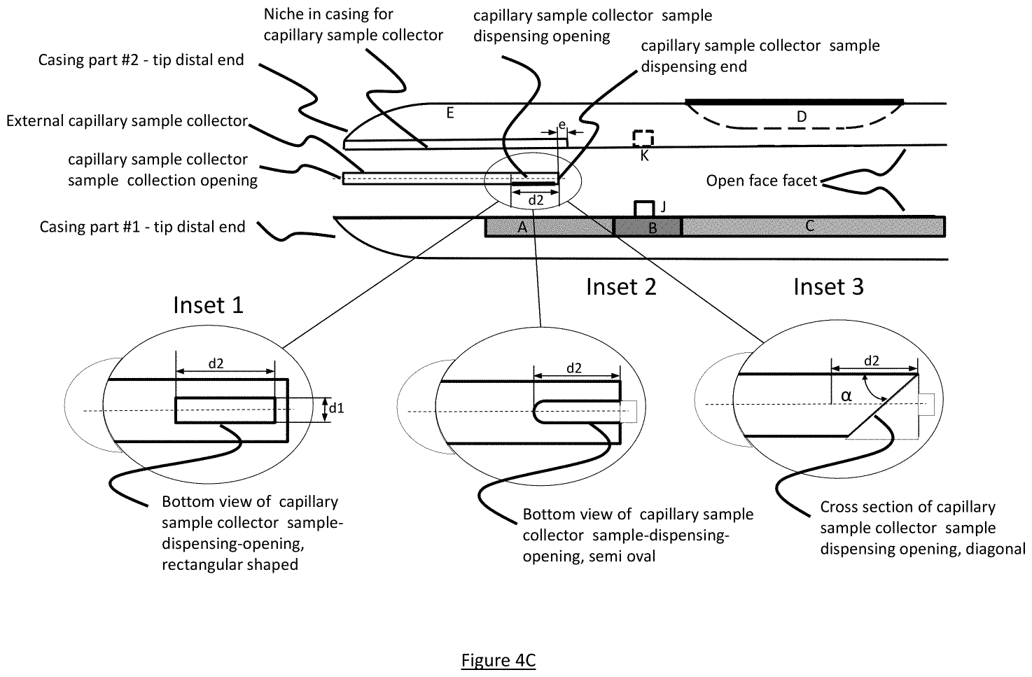

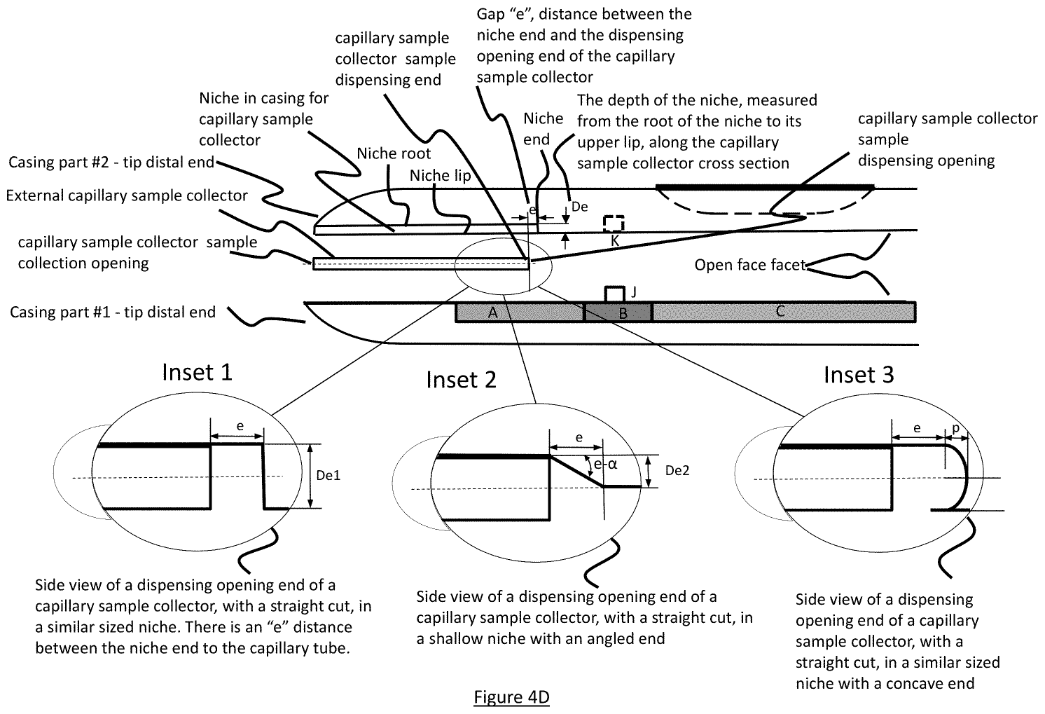

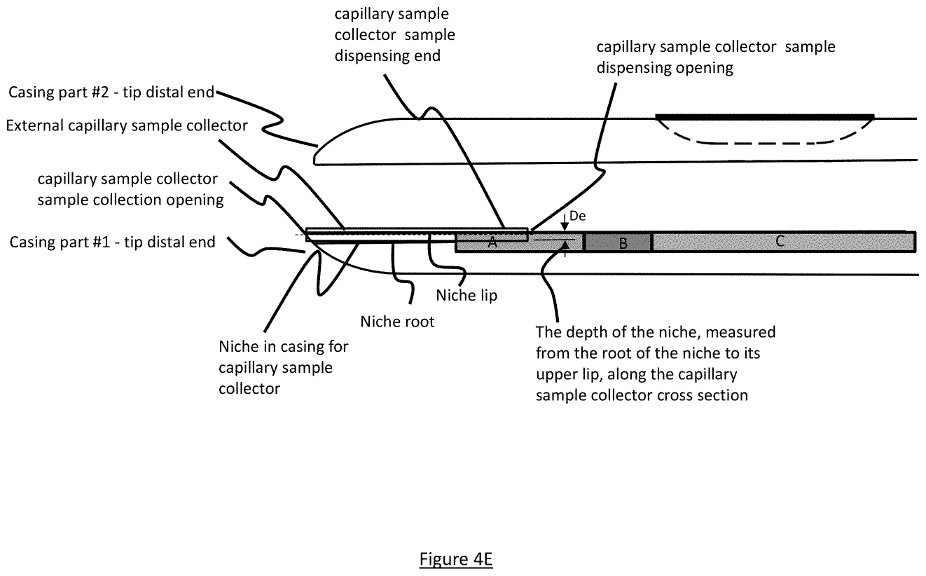

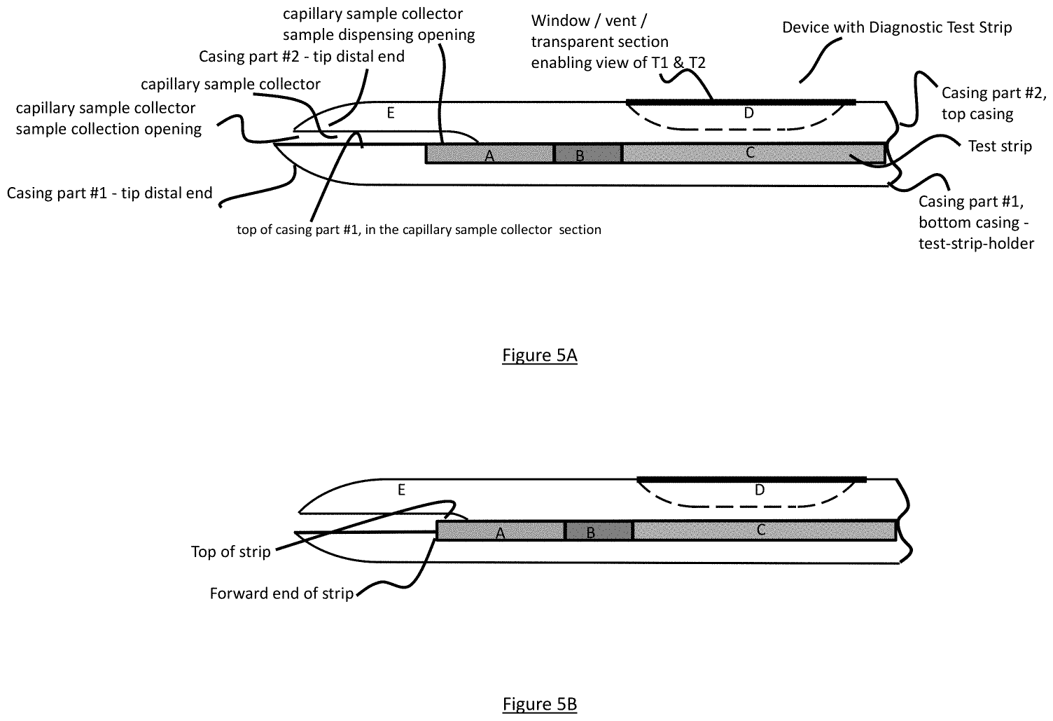

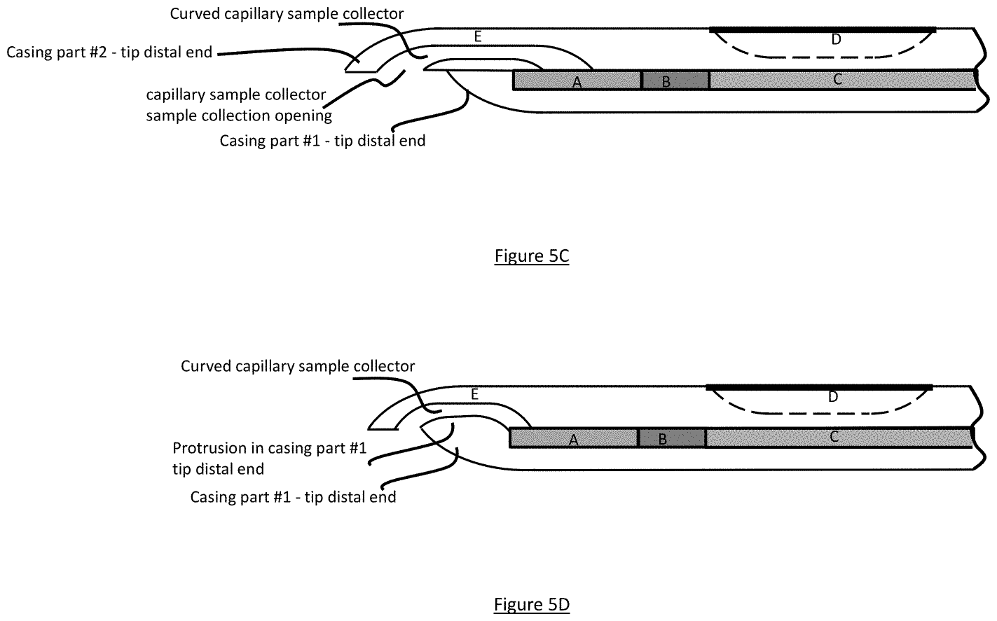

FIGS. 5A-5D show cross-sections of embodiments of casing device parts, including a diagnostic test strip. FIG. 5A includes labels for all elements of the casing device, while elements of the casing device that are identical to the labels in FIG. 5A are not again labeled in FIGS. 5B-5D.

FIG. 5A shows a longitudinal cross section of a casing including a bottom casing, part #1, and a top casing, part #2. The capillary sample collector is formed when the casing parts are assembled. In some embodiments, the capillary sample collector is linear.

In some embodiments, the capillary sample collector is formed from a groove or niche in the top casing, part #2, while bottom casing, part #1 is primarily flat. In some embodiments, the capillary sample collector is formed from a groove or niche in both the top casing, part #2 and bottom casing, part #1.

In some embodiments, the top of the test strip has the same height as the top of casing part #1, in the capillary sample collector section. In some embodiments, as shown in FIG. 5B, the top of the test strip is higher than the top of casing part #1, in the capillary sample collector section. In some embodiments, the top of the test strip is higher than the top of casing part #1 in the capillary sample collector section by less than 0.01 mm, or by less than 0.05 mm, or by less than 0.1 mm, or by less than 0.25 mm, or by less than 0.5 mm, or by less than 0.75 mm, or by less than, 1.0 mm, or by less than 1.5 mm, or by less than 2.0 mm, or by less than 3.0 mm.

In some embodiments, the forward end of the capillary sample collector sample dispensing opening is in line with the forward end of the strip. In some embodiments, the forward end of the capillary sample collector sample dispensing opening is within the sample pad A, behind the forward end of the strip.

In some embodiments, the distal ends of casing part #1 and casing part #2 are approximately one above the other. In some embodiments, the distal end of casing part #1 is forward of distal end of casing part #2 by less than 1.0 mm, or by less than 2.0 mm, or by less than 3.0 mm, or by less than 4.0 mm, or by less than 5.0 mm. In some embodiments, the distal end of casing part #1 is behind the distal end of casing part #2 by less than 5.0 mm, or by less than 4.0 mm, or by less than 3.0 mm, or by less than 2.0 mm, or by less than 1.0 mm.

In some embodiments the capillary sample collector sample collection opening faces upwards. In some embodiments, the capillary sample collector sample collection opening faces downwards.

FIG. 5C shows a longitudinal cross section of a casing including a bottom casing, part #1 and a top casing, part #2. The capillary sample collector is formed when the casing parts are assembled. In an embodiment, the capillary sample collector is curved.

In some embodiments, the capillary sample collector is formed within the top casing, casing part #2 while the bottom casing, part #1, is primarily flat. In some embodiments, the top of the test strip has the same height as the top of casing part #1 in the capillary sample collector section.

In some embodiments, the forward end of the capillary sample collector sample dispensing opening is in line with the forward end of the strip. In some embodiments, the forward end of the capillary sample collector sample dispensing opening, is within the sample pad A, behind the forward end of the strip.

In some embodiments, the distal end of top casing part #2 is positioned forward of the distal end of bottom casing part #1 by less than 0.1 mm, or by less than 0.5 mm, or by less than 1.0 mm, or by less than 2.0 mm, or by less than 3.0 mm, or by less than 4.0 mm, or by less than 5.0 mm.

In some embodiments, the capillary sample collector sample collection opening faces downwards.

FIG. 5D shows a longitudinal cross section of a casing including a bottom casing, part #1, and a top casing, part #2. The capillary sample collector is formed when the casing parts are assembled. In some embodiments, the capillary sample collector is curved.

In some embodiments, the capillary sample collector is formed from a groove or niche in the top casing, part #2, and a protrusion in casing part #1 tip distal end.

In some embodiments, the capillary sample collector is formed within the top casing, casing part #2, while the bottom casing, part #1, is primarily flat. In some embodiments, the top of the test strip has the same height as the top of casing part #1, in the capillary sample collector section.

In some embodiments, the forward end of the capillary sample collector sample dispensing opening is in line with the forward end of the strip. In some embodiments, the forward end of the capillary sample collector sample dispensing opening, is within the sample pad A, behind the forward end of the strip. In some embodiments, the distal end of top casing part #2 is forward of distal end of bottom casing part #1 by less than 0.1 mm, or by less than 0.5 mm, or by less than 1.0 mm, or by less than 2.0 mm, or by less than 3.0 mm, or by less than 4.0 mm, or by less than 5.0 mm.

FIGS. 6A and 6B show cross-sections of embodiments of a casing device, including parts of the casing device and a diagnostic test strip. FIG. 6A shows a longitudinal cross section of a casing including a bottom casing, part #1, and a top casing, part #2. In some embodiments, such as the embodiment shown in FIG. 6A, a gasket is positioned between the casing parts. In some embodiments, the gasket conforms with the sections of the top and bottom casings that it is in contact with. In some embodiments, the gasket fills cracks and voids between the assembled casing parts. It will be known to those of skill in the art that such cracks and voids may appear when mechanical parts are assembled, due to tolerances and surface finish of the casing parts, or because of design considerations. In some cases, when in contact with a fluid, the cracks and or voids will cause a capillary effect that forces the fluid to penetrate the void.

In some embodiments, the gasket includes a slot to accommodate a capillary sample collector. In some embodiments, the capillary sample collector is formed by the casing parts and the gasket. In some embodiments, the gasket material is in contact with the fluid flowing in the capillary sample collector.

In some embodiments, the gasket is made of a compliant material. In some embodiments, the gasket material includes at least one of the following: paper, rubber, silicone, metal, cork, felt, neoprene, nitrile rubber, fiberglass, polytetrafluoroethylene (otherwise known as PTFE, such as the material commercialized by Chemours of Wilmington, Del. under the trade name TEFLON), or a plastic polymer (such as polychlorotrifluoroethylene)

In some embodiments, the thickness of the gasket is more than 0.01 mm, or is more than 0.05 mm, or is more than 0.1 mm, or is more than 0.5 mm, or is more than 1.0 mm, or is more than 2.0 mm, or is more than 3.0 mm, or is more than 4.0 mm, or is more than 5.0 mm. In some embodiments, the thickness of the gasket is reduced (e.g., the gasket is compressed) by more than 1%, or by more than 5%, or by more than 10%, or by more than 15%, or by more than 20%, or by more than 25%, or by more than 35%, or by more than 50%, or by more than 60%, or by more than 75% after assembly. In some embodiments, the thickness of a portion of the gasket is reduced by more than 1%, or by more than 5%, or by more than 10%, or by more than 15%, or by more than 20%, or by more than 25%, or by more than 35%, or by more than 50%, or by more than 60%, or by more than 75% after assembly.

In some embodiments, a portion of the gasket is positioned between the casing parts in the casing tip area, where the casing tip area refers to the area from the forward end of the strip to the distal end of the casing device.

In some embodiments, a portion of the gasket is positioned in a cavity in the bottom casing, part #1. In some embodiments, a portion of the gasket is positioned in a cavity in the bottom casing, such that its outward facing surface is flush with the bottom casing highest point. In some embodiments, a portion of the gasket is positioned in a cavity in the bottom casing, part #1 such that its outward facing surface is above the bottom casing highest point.

In some embodiments, a portion of the gasket is positioned in a cavity in the top casing, part #2. In some embodiments, a portion of the gasket is positioned in a cavity in the top casing, such that its outward facing surface is flush with the top casing highest point. In some embodiments, a portion of the gasket is positioned in a cavity in the top casing, part #2 such that its outward facing surface is above the bottom casing highest point, wherein above implies that the thickness increases.

In some embodiments, a portion of the gasket is positioned in a cavity in the bottom casing, part #1, while a portion of the gasket is positioned in a cavity in the top casing, part #2.

In some embodiments, the cavity depth is less than 0.01 mm, or is less than 0.02 mm, or is less than 0.03 mm, or is less than 0.05 mm, or is less than 0.10 mm, or is less than 0.15 mm, or is less than 0.25 mm, or is less than 0.40 mm, or is less than 0.50 mm, or is less than 0.75 mm, or is less than 1.00 mm, or is less than 1.50 mm. In some embodiments, the cavity defines a location in which the gasket is positioned. In some embodiments, the cavity depth is 0.00 mm (within manufacturing tolerances).

FIG. 6B shows a longitudinal cross section of a casing including a bottom casing, part #1, and a top casing, part #2. In some embodiments, such as the embodiment shown in FIG. 6B, a strip gasket is positioned between the casing parts.

In some embodiments, a strip gasket's length dimension is significantly larger than its width or diameter. In some embodiments, the strip gasket is a strip whose width is significantly larger than its thickness, such as a masking tape. In some embodiments, the strip gasket is a pre-pressed strip gasket. In some embodiments, the strip gasket is a pre-pressed strip gasket that has a round profile, such as a sealing material dispensed form a tube.

In some embodiments, a portion of the strip gasket is positioned between the casing parts in the casing tip area, where the term “casing tip area” refers to the area from the forward end of strip to the distal end of the casing device. In some embodiments, a portion of the strip gasket is positioned in the casing tip area that may come in contact with fluid. In some embodiments, a portion of the strip gasket is positioned in the casing tip area that may come in contact with sample fluid or reagent fluid.

In some embodiments, a portion of the strip gasket is positioned on a portion of the rim of the top casing part. In some embodiments, a portion of the strip gasket is positioned on a portion of the casing tip of the top casing part. In some embodiments, a portion of the strip gasket is positioned on a portion of the rim of the bottom casing part. In some embodiments, a portion of the strip gasket is positioned on a portion of the casing tip of the bottom casing part.

In some embodiments, the gasket also functions as an adhesive. In some embodiments, the gasket also functions as an adhesive that connects the casing parts and also seals cracks and voids.

In some embodiments, an exemplary sample collector such as those described above is used in connection with an exemplary reagent tube. In some embodiments, the tube serves as the reservoir for the reagent that drives the sample into the lateral flow test strip for analysis. In some embodiments, the tube containing the reagent is sealed by a foil preventing reagent evaporation. In some embodiments, the tube containing the reagent is sealed by a cap preventing reagent evaporation. In some embodiments, the reagent is composed of a buffer that helps to solubilize the sample, facilitating uniform capillary flow along the lateral flow membrane.

In some embodiments, the reagent tube is configured to form an air-tight seal with the device casing, allowing for air pressure buildup inside the tube. In some embodiments, the reagent tube is configured such that air pressure buildup occurs after liquid contact between the capillary sample collector and the reagent takes place, thereby ensuring that air bubbles are not introduced into the capillary sample collector which may interfere with sample transfer to the sample pad of the test strip.

In some embodiments, reagent tube includes a porous element (e.g., a sponge) positioned at the bottom of the tube and having the reagent absorbed therein. In some embodiments, providing the reagent that is absorbed into a porous element ensures that the reagent is confined to the bottom of the tube, further assuring that fluid contact is made between the capillary sample collector and the reagent regardless of gravitational forces. As a result, in such embodiments, the rapid diagnostic test may be performed in any position, either vertically or horizontally.

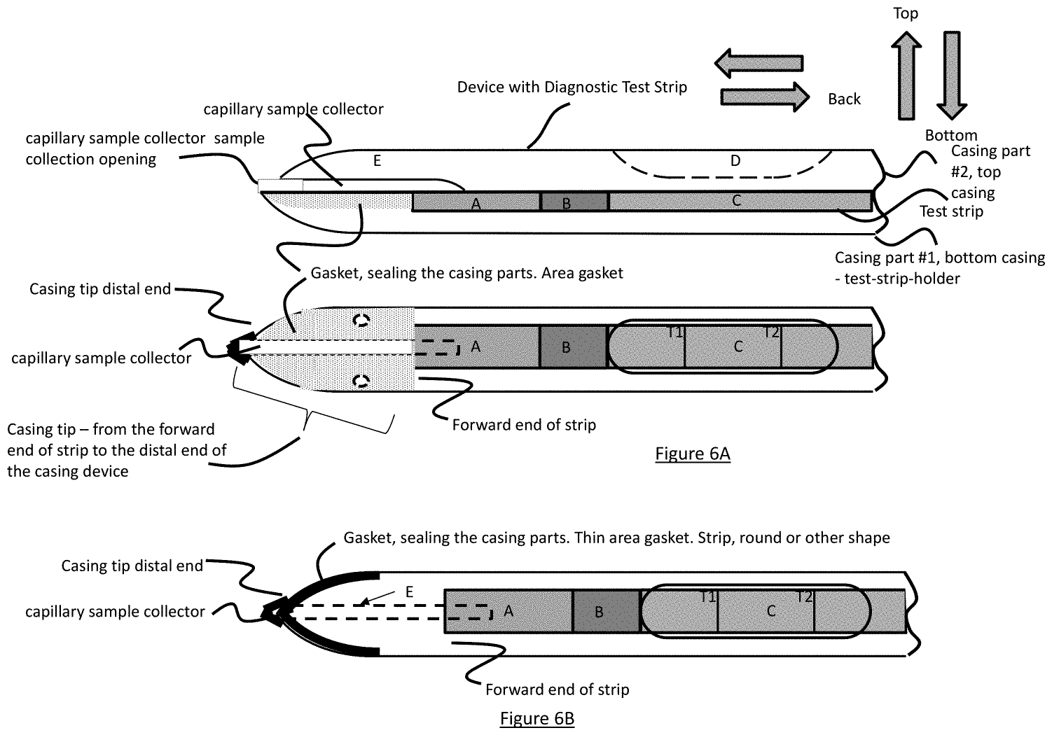

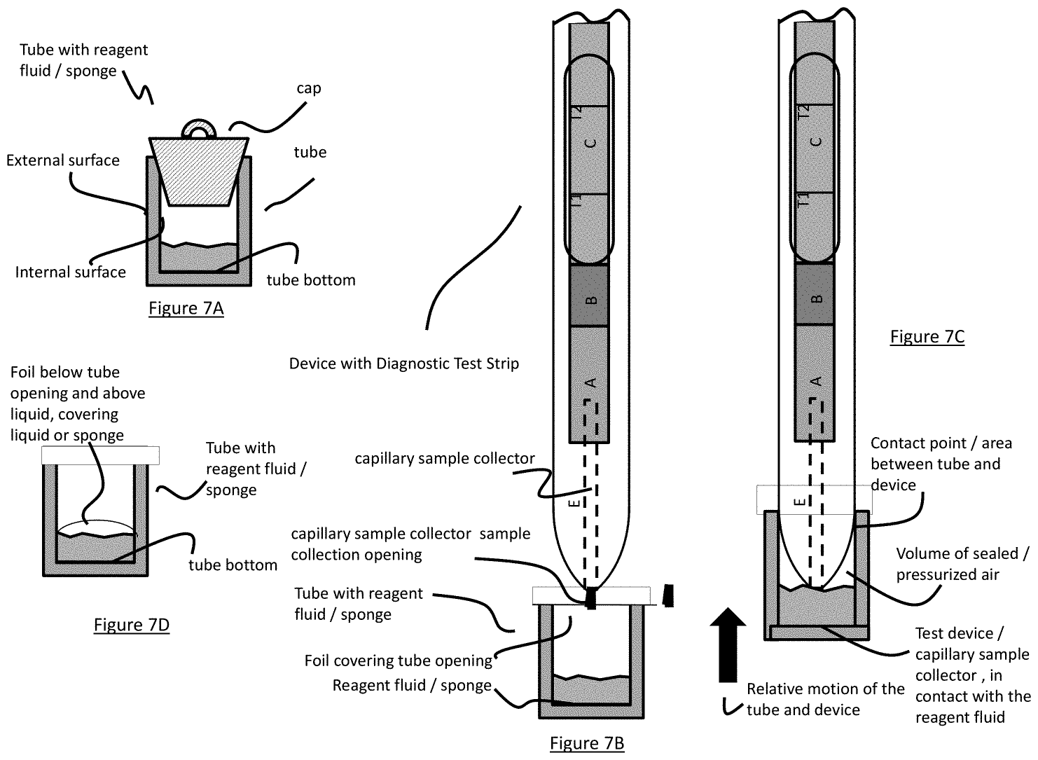

Figures FIGS. 7A-7D show embodiments of a reagent tube and use thereof. In some embodiments, the reagent tube serves as the reservoir for the reagent, which is configured to drive the sample into the lateral flow test strip for analysis. In some embodiments, such as shown in FIGS. 7B and 7D, the tube containing the reagent is sealed by a foil. In some embodiments, such as shown in FIG. 7A, the reagent tube is sealed by a cap. In some embodiments, the reagent tube is sealed by another type of seal. In some embodiments, the foil, cap, or other seal is configured to prevent reagent evaporation. In some embodiments, the reagent is composed of a buffer that helps solubilize the sample and allow uniform capillary flow along the lateral flow membrane.

FIG. 7A shows the elements of an exemplary reagent tube that is sealed by a cap. In some embodiments, the elements include an external surface, an internal surface, and a bottom of the internal surface of the tube.

FIG. 7B shows a device casing prior to being inserted into a reagent tube with a foil covering the tube opening. FIG. 7C shows a device casing being inserted into a reagent tube. In some embodiments, the reagent tube forms an air-tight seal with the device casing, allowing for air pressure buildup inside the tube. In some embodiments, the device casing and the reagent tube are configured such that air pressure buildup occurs after a liquid contact between the capillary sample collector and the reagent takes place, thereby assuring that air bubbles are not introduced into the capillary sample collector. It will be apparent to those skilled in the art that air bubbles in the capillary sample collector may interfere with sample transfer to the sample pad (A) of the test strip.

In some embodiments, the sample-acquiring end of the casing device, also referred to as the casing tip distal end, has a sharp tip that can penetrate a foil that covers an opening of a tube containing the reagent. In some embodiments, following the penetration of the foil, the device's tip is brought into contact with the liquid reagent, allowing the reagent to enter the sample acquiring end of the capillary sample collector.

FIG. 7D shows a reagent tube with a foil covering the fluid in the tube. In some embodiments, the foil is positioned above the reagent liquid such that there is a small amount of gas above the liquid. In some embodiments, the foil is positioned above the fluid and below the tube opening. In some embodiments, the foil is dome shaped. In some embodiments, the foil is dome shaped and encloses pressurized (e.g., at above atmospheric pressure) volume of air. In some embodiments, a liquid storage blister pouch is positioned at the bottom of the tube, such that the foil is the blister pouch cover.

FIGS. 8A and 8B show an embodiment in which a liquid reagent is absorbed into a porous element (e.g., a sponge) that is placed at the bottom of the tube, that is, the far end of the tube from the tube's open end. FIG. 8A shows the sample collector and the reagent tube prior to the combination thereof, while FIG. 8B shows the sample collector and the reagent tube in use. In some embodiments, positioning of the reagent absorbed into a porous element that is positioned at the bottom of the tube ensures that the reagent is confined to the bottom of the tube, further assuring that fluid contact is made between the capillary sample collector and the reagent regardless of gravitational forces, i.e., the rapid diagnostic test may be performed at any position, vertically, horizontally (as shown in FIG. 8B), or at any angle.

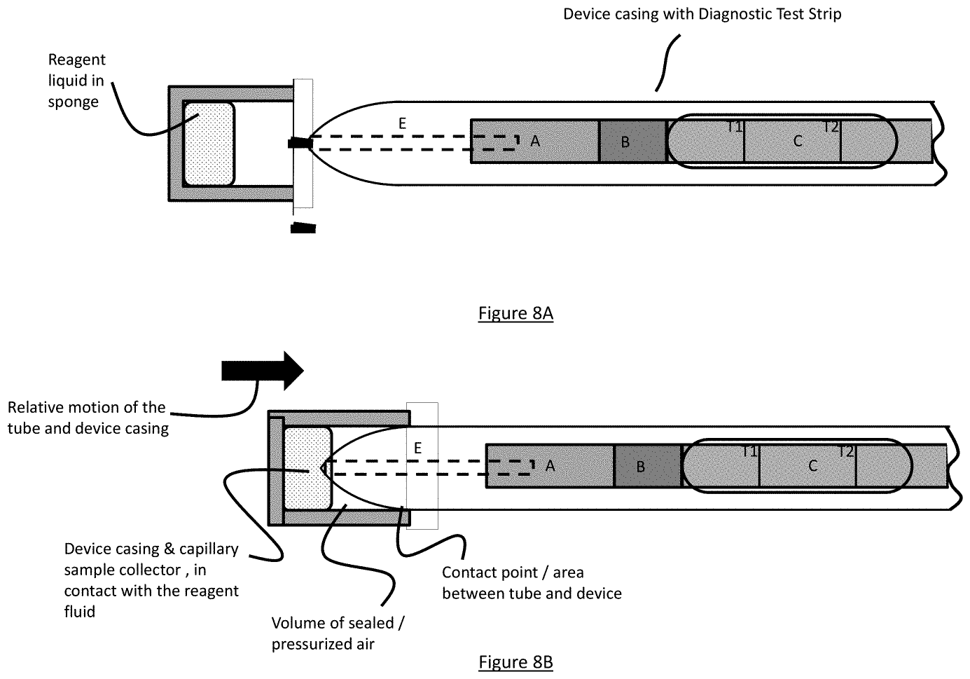

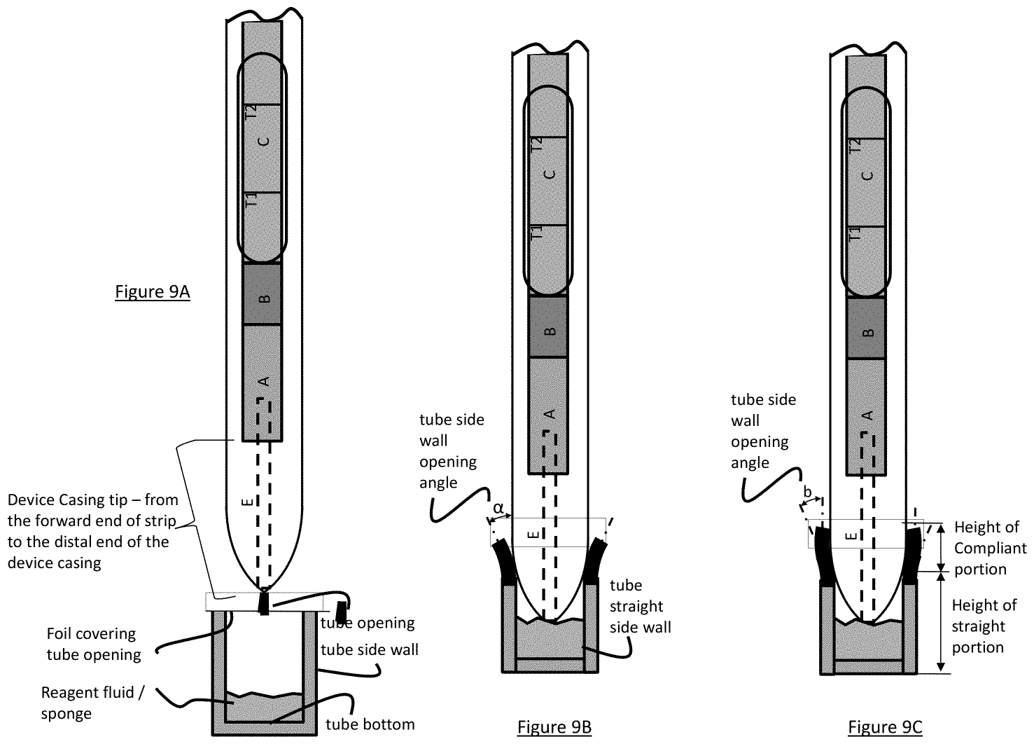

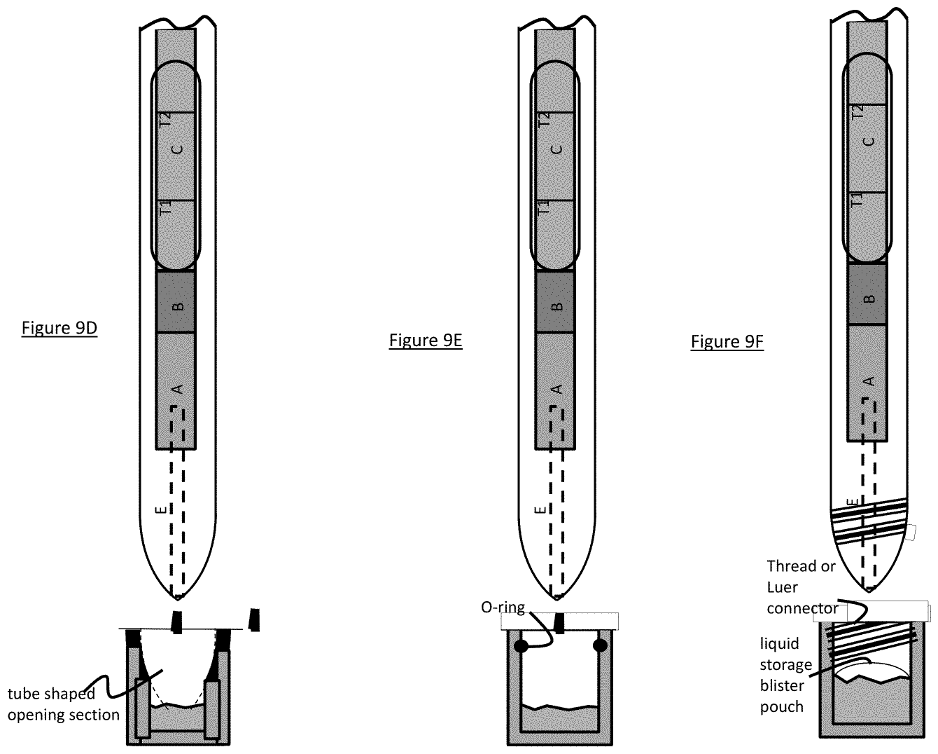

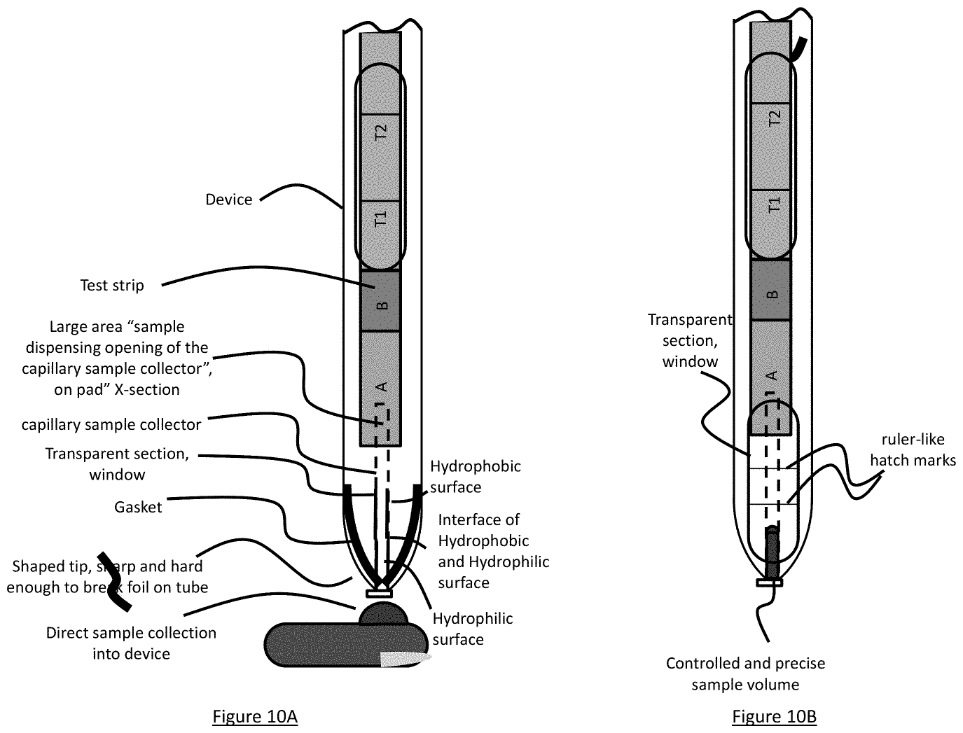

FIGS. 9A-9F show embodiments of reagent tubes and device casings configured to form a seal upon the insertion on the device casing into the reagent tube.

FIG. 9A shows a tube used as a reservoir for the reagent fluid. As shown in FIG. 9A, the tube has a bottom aspect, side walls/surfaces, and an opening. In some embodiments, the opening is sealed. In some embodiments, the opening is sealed with a foil. In some embodiments, the opening is sealed with a cap. In some embodiments, the opening is closed with a foil or cap. In some embodiments, the reagent fluid is soaked into a sponge. In some embodiments, the reagent fluid is soaked into a sponge that is placed at the bottom of the tube.

As used herein, the device casing tip refers to the casing section from the forward end of strip to the distal end of the device casing.

In some embodiments, the diameter of the device casing tip is slightly larger than the diameter of the tube. In some embodiments, the diameter of the device casing tip is slightly larger than the diameter of the tube such that insertion of the device casing tip into the tube creates an interface fit. In some embodiments, the tube material is compliant (e.g., elastic, deformable, etc.), such that the interface fit creates a seal, without damaging the device casing or the tube. In some embodiments, the device casing material is compliant such that the interface fit creates a seal, without damaging the device casing or the tube. In some embodiments, the device casing tip section is made of a material that is compliant such that the interface fit creates a seal, without damaging the device casing or the tube.

FIG. 9B shows a tube used as a reservoir for the reagent fluid, wherein a section of the tube walls in proximity to the tube opening is angled outwards. In some embodiments, the inclusion of a section of the tube walls that is angled outwards increases the diameter of the tube opening. In some embodiments, the increased diameter serves as a guide to the device casing tip during insertion.

In some embodiments, the tube side wall includes a straight section and a section that is at an angle, a. In some embodiments, the angle α is less than 45°, or is less than 40°, or is less than 30°, or is less than 20°, or is less than 10°, or is less than 5°, or is less than 3°. In some embodiments, the opening diameter is larger than the diameter of the straight section by less than 1%, or by less than 2%, or by less than 4%, or by less than 7%, or by less than 10%, or by less than 15%, or by less than 20%, or by less than 30%, or by less than 40%.

FIG. 9C shows a tube used as a reservoir for the reagent fluid, wherein a section of the tube walls in proximity of the tube opening is compliant and therefore conforms to the shape of the device casing being inserted. In some embodiments, the inclusion of a compliant section that conforms to the shape of the device casing tip increases the contact area that forms the seal.

In some embodiments, the section of the tube walls in proximity to the tube opening is compliant due to material properties. In some embodiments, the section of the tube walls in proximity to the tube opening includes design features that make it compliant. In some embodiments, the tube opening has a diameter that is slightly smaller than that of the device tip.

FIG. 9D shows a tube used as a reservoir for the reagent fluid, wherein a section of the tube walls in proximity of the tube opening has a shape that is similar to the shape of the device casing tip. In some embodiments, the inclusion of a section of the tube walls in proximity to the tube opening of similar shape to the device casing tip increases the contact area that forms the seal.

FIG. 9E shows a tube used as a reservoir for the reagent fluid, wherein an O ring is positioned in the tube walls in proximity to the tube opening. In some embodiments, the inclusion of an O-ring in the section of the tube walls in proximity to the tube opening provides a contact area with the device casing tip and facilitates formation of an airtight seal. In some embodiments, during insertion of the device casing into the tube, the O-ring deforms such that the contact area between the O-ring and the device casing tip increases.

In an embodiment, the O-ring cross section is round. In an embodiment, the O-ring cross section is not round. In an embodiment, the O-ring cross section has a square, rectangular, trapezoidal, oval or other shape.

FIG. 9F shows a tube used as a reservoir for the reagent fluid, wherein the internal surface of the tube in proximity of the tube opening includes an internal screw thread. In some embodiments, such as the embodiment shown in FIG. 9F, the device casing includes a reciprocal external screw thread that is configured to mate with the internal screw thread of the tube. In some embodiments, the tube is configured to be connected to the device casing by aligning the internal and external threads and rotating the tube and the device casing with respect to one another. In some embodiments, the screw threads used to connect the device casing to the tube form a contact area that forms a seal. In some embodiments, the screw threads used to connect the device casing to the tube limit the motion of the device casing in the tube, such that it cannot exceed the thread length. In some embodiments, the screw threads used to connect the device casing to the tube provide a predetermined insertion depth. In some embodiments, the screw threads used to connect the device casing to the tube are configured to reduce the rate at which reagent is driven from the reagent tube into the capillary tube, thereby reducing the rate of fluid flow in the capillary sample collector, which helps control reagent flow, further preventing reagent overflow into the test strip. In some embodiments, the screw threads slow down lateral advancement of the capillary tube into the porous element that holds the reagent, thereby slowing down the rate of reagent flow into the capillary tube and from there to the sample pad of the test strip.

In some embodiments, the device end includes a Luer connector. In some embodiments, a Luer connector is integrated into the device end. In some embodiments, the tube opening includes a Luer connector. In some embodiments, a Luer connector is integrated into the tube.

In an embodiment, the bottom of the tube includes a liquid storage blister pouch. In an embodiment, a liquid storage blister pouch is positioned at the bottom of the tube. In some embodiments, a liquid storage blister pouch is integrated into the bottom of the tube.

In some embodiments, following filling of the capillary sample collector by the required biological sample volume, a reagent challenge is introduced to the sample-loading end of the capillary sample collector. In some embodiments, the reagent challenge forces the entire captured volume of bodily fluid to flow through the unfilled section of the capillary sample collector, further flowing into the sample pad of the lateral flow test strip.

In some embodiments, the reagent challenge is assisted by a positive pressure created when the tip of the device is inserted into the tube carrying the reagent, resulting in formation of an airtight seal between the device tip and the rim of the reagent tube, as will be described below with reference to FIGS. 10A-10D. In some embodiments, this positive pressure acts to induce the flow of the reagent from its container into the capillary sample collector that carries the biological fluid sample. In some embodiments, movement of the reagent into the capillary sample collector, in turn, forces the biological sample past the hydrophobic block in the sample collector region and onto the sample pad of the test strip. In some embodiments, the biological sample does not become diluted by the reagent, thereby optimizing sample loading into the sample pad and its chromatographic flow along the test strip.

In some embodiments, the reagent container and the device casing are configured such that fluid contact is first made between the device tip (which carries the sample-filled capillary sample collector) and the reagent in the tube, before the airtight seal is formed. In some embodiments, this sequence ensures that no air bubbles, which may prevent proper sample loading to the test strip, are formed at the capillary sample collector's opening.

FIGS. 10A-10D illustrate use of an exemplary device to collect a sample and transfer the sample to the sample pad of a test strip. FIG. 10A shows the use of an exemplary device for direct sample collection. In some embodiments, a device includes a test strip and a capillary sample collector. In some embodiments, the capillary sample collector includes a section in which its inner surface has hydrophilic properties. In some embodiments, the distal end of the section with the hydrophilic properties coincides with the casing distal end. In some embodiments, the distal end of the section with the hydrophilic properties is in proximity to the capillary sample collector distal end.

In some embodiments, the capillary sample collector includes a section in which its inner surface has hydrophobic properties. In some embodiments, the hydrophobic section is adjacent to the hydrophilic sections, and is positioned towards the capillary sample collector sample dispensing opening as compared to the portion of the capillary sample collector with hydrophilic properties. In some embodiments, there is an interface between the hydrophobic surface and the hydrophilic surface.

In some embodiments, the device casing includes a transparent section that functions like a window, allowing the capillary sample collector to be viewed. In some embodiments, the transparent section enables a user to view the fluid in the capillary sample collector. In some embodiments, there are ruler-like hatch marks on the transparent section. In some embodiments, a user viewing the fluid in the capillary sample collector through the transparent section with hatch marks can gauge the volume of fluid in the sample collector.

In some embodiments, the device is designed such that the sample dispensing opening of the capillary sample collector overlaps the test strip sample pad A.

In some embodiments, the device casing includes a gasket positioned in the device casing tip. In some embodiments, the tip is sharp enough to break the foil sealing the tube with reagent fluid.

FIG. 10B shows a device used for direct sample collection after a volume of sample fluid has been collected. In some embodiments, the combination of the hydrophilic portion and the hydrophobic portion allows the collection of a precise volume of sample fluid. In some embodiments, the fluid collected is forced up the capillary sample collector by the capillary forces of the hydrophilic surface.

In some embodiments, the fluid stops filling the capillary collector when it reaches the hydrophobic surface. In some embodiments, the fluid stops filling the capillary collector when it reaches the interface between the hydrophobic surface and the hydrophilic surface. In some embodiments, the length of the hydrophilic section is designed such that a desired volume of sample fluid can be collected. In some embodiments, the interface between the hydrophobic surface and the hydrophilic surface is designed such that a desired volume of sample fluid can be collected.

FIG. 10C shows a sample collection device that is inserted into a tube containing a reagent fluid. As shown in FIG. 10C, the device casing tip is in contact with the reagent fluid and a seal is formed between the device casing and the tube.

In some embodiments, a force F is applied between the device and tube in a direction so as to further the insertion of the device into the tube. In some embodiments, continued insertion past the point that the device has sealed the tube results in a positive pressure within the tube. In some embodiments, the insertion length defines the pressure magnitude.

FIG. 10D shows the operation of the positive pressure within the tube, above the fluid and below the device tip, so as to force the reagent fluid into the capillary sample collector. In some embodiments, the reagent fluid being forced up the capillary sample collector drives the sample fluid past the hydrophobic section and onto the test device sample area A.

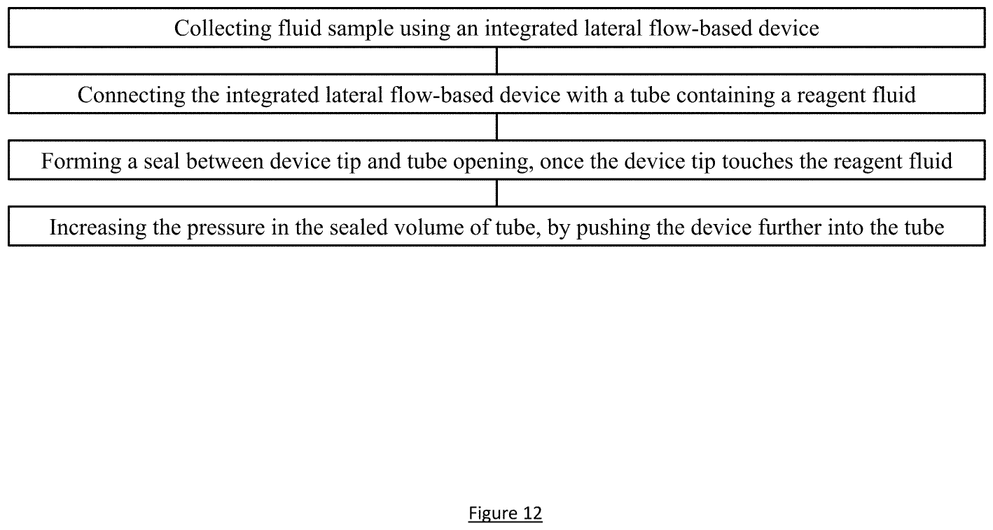

FIG. 11 shows an exemplary method for collecting and diagnosing fluid samples. The method of FIG. 11 includes the following steps:

1. Collecting fluid sample using an integrated lateral flow-based device

2. Connecting the integrated lateral flow-based device with a tube containing a reagent fluid

3. Forming a seal between device tip and tube opening, after the device tip touches the reagent fluid

4. Increasing the pressure in the sealed volume of tube by pushing the device further into the tube

5. Driving the reagent fluid up the capillary sample collector

6. Driving the fluid sample past the hydrophobic coating block and onto the test strip.

FIG. 12 shows an exemplary method for collecting and diagnosing fluid samples. The method of FIG. 12 includes the following steps:

1. Collecting fluid sample using an integrated lateral flow-based device

2. Connecting the integrated lateral flow-based device with a tube containing a reagent fluid

3. Forming a seal between device tip and tube opening, once the device tip touches the reagent fluid

4. Increasing the pressure in the sealed volume of tube, by pushing the device further into the tube

In some embodiments, the method of FIG. 12 is performed in a manner such that the increased pressure drives the reagent fluid up the capillary sample collector. In some embodiments, the method of FIG. 12 is performed in a manner such that the reagent fluid that is driven up the capillary sample collector drives the sample fluid past the hydrophilic coating block and into the test strip for analysis.

As used herein, the term “absorbent pad” (alternately referred to as a “fluid wick”) refers to a highly-absorbent material which serves to absorb fluid from the lateral flow membrane's distal end, thereby assisting with fluid flow along the lateral flow membrane and collecting fluid. The role of the absorbent pad is to wick the fluid through the membrane and to collect the processed liquid. An example of a common absorbent pads is one made of cellulose filters.

As used herein, the term “backing element” refers to non-porous plastic strip, usually made of polyester or other durable plastic material. Examples of common backings are 2-mil and 4-mil polyester films.

As used herein, the term “capillary sample collector” refers to a tube or porous element that serves to collect a certain volume of biological sample in the range of 1-100 microliters. Control of capillary forces may be achieved by tailoring the surface energy of this element, such that capillary fluid rise will only occur on a high surface energy (hydrophilic) segments and be prevented in low surface energy (hydrophobic) ones.

As used herein, the term “capture molecule” refers to a molecule, usually an antibody or antigen, that is immobilized by printing on the lateral flow membrane and serving to capture the target analyte in the form of its complex with the detector molecule.

As used herein, the term “casing” refers to the device parts that house the test strip and the capillary sample collector and brings them together in fluid contact, allowing the sample collected by the capillary sample collector to flow into the sample pad of the test strip, further allowing reagent fluid to propagate the sample and its components (including the target analyte) through the conjugate pad and further into the lateral flow test strip.

As used herein, the term “conjugate pad” refers to an element that is used for reversibly absorbing the detector molecule, along other ingredients such as detergents, carbohydrates, etc., keeping them functionally stable until the test is performed. When the conjugate particles, used to label the detector molecule, are dried in the presence of sugar, the sugar molecules form a layer around them stabilizing their biological structures. When the sample enters the conjugate pad, the sugar molecules rapidly dissolve carrying the detector molecule conjugates into the fluid stream, allowing the detector molecule conjugates to bind the analyte in the sample.

As used herein, the terms “control lines” and “test lines” refer to lines of non-specific capture molecules and analyte-specific capture molecules, respectively, printed at different locations across the test strip, for capturing target analyte-detector molecule complexes at the test line and non-specific binding of a labelled reagent at the control line, allowing clear visualization of non-specific (control) and specific (test) analyte binding accordingly.

As used herein, the term “detector molecules” refers to various types of detector reagents that can be used for the visualization of a signal. The most used labels in commercially available tests are latex beads and colloidal gold particles. Other possibilities include enzyme conjugates, other colloidal metals, fluorescent particles, and magnetic particles. These labeled antigen or antibody molecules bind the target analyte with sufficient sensitivity (affinity) and specificity to allow target analyte visualization once captured by the capture molecule on the test strip. One of the most important features of the particles used for labeling is that their population is monodisperse with consistency of size and spherical shape.

As used herein, the term “lateral flow membrane” refers to a membrane, often composed of nitrocellulose or another inert porous material, with pore size in the range of several microns, allowing effective capillary flow of fluid. This membrane may be, for example, 3-5 mm wide, 60-80 mm long and 0.2 mm thick. It is immobilized with capture molecules in the forms of 0.1-1.0 mm wide stripes printed at specific locations perpendicular to the direction of lateral flow over the membrane, allowing capture of the analyte-detector molecule complex at the test line and labeled non-specific molecule at the control line.

As used herein, the term “observation window” refers to a transparent window or opening in the casing immediately above the control and test lines on the lateral flow membrane, allowing their visualization.

As used herein, the term “porosity” refers to a measure of the void spaces in a material and is a fraction of the volume of voids over the total volume of the material. In some cases, porosity is represented over a range between 0 and 1. In some embodiments, porosity is represented as a percentage between 0% and 100%. In some cases, porosity is measured based on the “accessible void”, which the total amount of void space accessible from the surface of the material.

As used herein, the term “reagent” refers to the buffer or other aqueous solution used to drive the analyte along the test strip by capillary flow to dissolve and react with the detection molecule in the sample pad, forming complexes that further reach and interact with the capture molecule(s) in the test and control lines on the lateral flow membrane.

As used herein, the term “reagent tube” refers to a tube or other container containing a reagent.

As used herein, the term “sample” refers to any liquid biological material, including, but not limited to, blood (e.g., serum, plasma, etc.), urine, saliva, mucosal fluid, etc. It also includes other biological material, such as feces, that can be suspended or dissolved in liquid for the purpose of analysis

As used herein, the term “sample pad” (A) refers to a location where the biological fluid sample is being adsorbed by the test strip in preparation for its chromatography on the lateral flow membrane. It is usually made of non-binding fibrous material, such as cellulose or fiberglass, and used for filtering out debris, fat and other contaminants that may affect test strip performance. The sample pad can be used to perform multiple tasks, foremost of which is to promote the even and controlled distribution of the sample onto the conjugate pad. It may also control the rate at which liquid enters the conjugate pad, preventing flooding of the device. When impregnated with components such as proteins, detergents, viscosity enhancers, and buffer salts, the sample pad can also be used to: 1. Increase sample viscosity (modulate flow properties), 2. Enhance the ability of the sample to solubilize the detector reagent, 3. Prevent the conjugate and analyte from binding non-specifically to any of the downstream materials, 4. Modify the chemical nature of the sample so that it is compatible with immunocomplex formation at the test line, 5. Promote even flow of the sample along the membrane.

As used herein, the term “sorptivity” refers to a measure of lateral flow rate through a porous medium, which depends on several factors, including pore size and structure, building material's surface energy, etc.

As used herein, the term “target analyte” refers to the analyte or biomarker present in the sample that is to be detected by the test strip at sufficient sensitivity and specificity.

As used herein, the term “test strip” refers to a lateral flow test strip, commonly used in rapid diagnostic tests, composed of a sample pad (A), conjugate pad (B), lateral flow membrane (C), and a fluid wick, usually laminated in this order and glued on top of a plastic backing element.

In some embodiments, a device as described herein is configured to produce a fluid flow rate of less than 1 microliter per minute, or less than 2 microliters per minute, or less than 5 microliters per minute or less than 10 microliters per minute, or less than 20 microliters per minute, or less than 25 microliters per minute, or less than 30 microliters per minute, or less than 35 microliters per minute, or less than 40 microliters per minute, or less than 45 microliters per minute, or less than 50 microliters per minute.

In some embodiments, a device as described herein is configured to collect a volume of a sample that is from 5 μL to 75 μL, or from 5 μL to 65 μL, or from 5 μL to 55 μL, or from 5 μL to 45 μL, or from 5 μL to 35 μL, or from 5 μL to 25 μL, or from 5 μL to 15 μL, or from 15 μL to 75 μL, or from 15 μL to 65 μL, or from 15 μL to 55 μL, or from 15 μL to 45 μL, or from 15 μL to 35 μL, or from 15 μL to 25 μL, or from 25 μL to 75 μL, or from 25 μL to 65 μL, or from 25 μL to 55 μL, or from 25 μL to 45 μL, or from 25 μL to 35 μL, or from 35 μL to 75 μL, or from 35 μL to 65 μL, or from 35 μL to 55 μL, or from 35 μL to 45 μL, or from 45 μL to 75 μL, or from 45 μL to 65 μL, or from 45 μL to 5 μL, or from 55 μL to 75 μL, or from 55 μL to 65 μL, or from 65 μL to 75 μL.

In some embodiments, the linear velocity of fluid flowing along the lateral flow membrane is less than 5 millimeters per minute, or is less than 6 millimeters per minute, or is less than 7 millimeters per minute, or is less than 8 millimeters per minute, or is less than 9 millimeters per minute, or is less than 10 millimeters per minute.

In some embodiments, a device as configured herein is configured to operate at room temperature, or at a temperature of less than 5 degrees Celsius, or at a temperature of less than 10 degrees Celsius, or at a temperature of less than 15 degrees Celsius, or at a temperature of less than 20 degrees Celsius, or at a temperature of less than 25 degrees Celsius, or at a temperature of less than 30 degrees Celsius, or at a temperature of less than 35 degrees Celsius, or at a temperature of less than 40 degrees Celsius.

In some embodiments, a device as described herein is configured to provide test results, from the end of sample preparation to results observation, in 15 mins or less. In some embodiments, this duration includes 1-2 minutes for collecting the blood sample (depending on the lancet and patient/physician skills), 1-2 minutes for test initiation, including reagent challenge, and 5-10 minutes for lateral flow results to appear

In some embodiments, a device as described herein is configured to provide test results that remain visible for at least ten minutes. In some embodiments, a device as described herein is configured to provide test results that remain visible for between ten minutes and two days. In some embodiments, results should typically be read within 20 minutes of test performance. In some embodiments, test results remain stable and visible for a few hours.

In some embodiments, a device as described herein is configured to provide a lateral flow rate in the range of 5-10 millimeters per minute along the lateral flow membrane. It will be known to those of skill in the art that flow rate determines sensitivity, where a higher flow rate produces a test having lower sensitivity. It will be further known to those of skill in the art that lateral flow rates are determined at least by lateral flow membrane sorptivity.

In some embodiments, a device as described herein has a shelf life of at least 24 months when stored at between 4° C. and 30° C.

While a number of embodiments of the present invention have been described, it is understood that these embodiments are illustrative only, and not restrictive, and that many modifications may become apparent to those of ordinary skill in the art. Further still, the various steps may be carried out in any desired order (and any desired steps may be added and/or any desired steps may be eliminated).

What is claimed is:

1. A device, comprising:
a casing first part;
a casing second part;
a test strip having a sample pad; and
a capillary sample collector, wherein the capillary sample collector has (a) an open distal end configured to collect a fluid sample by capillary action and (b) an open proximal end configured to dispense the fluid sample therefrom,
wherein, the device is assembled such that the casing first part and the casing second part are joined,
wherein a distal end of the casing first part and a distal end of the casing second part are sealed together in a fluid-tight manner,
wherein the sample pad of the test strip is positioned in proximity to the capillary dispensing end,
wherein the dispensing end of the capillary sample collector is positioned such that a dispensed fluid sample will be dispensed onto the sample pad of the test strip, and
wherein a dispensing angle between the dispensing end of the capillary sample collector and the sample pad of the test strip is less than 10 degrees.

2. The device of claim 1, wherein the capillary sample collector is segmented into at least two length segments.

3. The device of claim 2, wherein a first length segment of the capillary sample collector includes a hydrophilic internal wall, and wherein a second length segment of the capillary sample collector includes a hydrophobic internal wall.

4. The device of claim 3, wherein the first length segment of the capillary sample collector is distally positioned so as to allow for fluid sample uptake by capillary forces, and wherein the second length segment of the capillary sample collector is proximally located so as to restrict an amount of fluid collected by the capillary sample collector.

5. The device of claim 4, wherein a length of the first length segment of the capillary sample collector is configured to enable collection of a predefined volume of fluid.

6. The device of claim 5, wherein the predefined volume is less than 100 microliters.

7. The device of claim 2, wherein a first length segment of the capillary sample collector includes a hydrophilic internal wall, and wherein a second length segment of the capillary sample collector includes a hydrophilic internal wall, and wherein the capillary sample collector further comprises an air gap positioned between the first length segment and the second length segment.

8. The device of claim 1, wherein the capillary sample collector has a hydrophilic internal wall, wherein the capillary sample collector forms a 90° angle at the open proximal end, wherein the device includes an air gap between open proximal end of the capillary sample collector and the sample pad of the test strip, and wherein the open proximal end of the capillary sample collector and the air gap cooperate to act as a burst valve at the open proximal end of the capillary sample collector.

9. The device of claim 1, wherein the capillary sample collector is segmented into at least two length segments, wherein a first length segment of the capillary sample collector includes a hydrophilic internal wall, wherein a second length segment of the capillary sample collector includes a hydrophobic internal wall, and wherein a volume of the open proximal end is smaller than a volume of the first length segment of the capillary sample collector, whereby the device is configured not to dilute a sample prior to the sample reaching the sample pad.

10. The device of claim 1, wherein the device is configured such that a portion of a sample fluid dispensed onto the test strip in not diluted.

11. The device of claim 10, wherein the portion is less than 50%.

12. The device of claim 1, wherein, the capillary sample collector is made of a different material than the casing first part.

13. The device of claim 12, wherein the capillary sample collector is made of glass.

14. The device of claim 12, wherein the capillary sample collector is made of a polymer.

15. The device of claim 12, wherein a plane defined by the capillary sample collector opening is perpendicular to an axis of the capillary sample collector.

16. The device of claim 12, wherein a plane defined by the capillary sample collector opening is oriented at an angle of less than 90° to an axis of the capillary sample collector.

17. The device of claim 1, wherein the casing first part includes a groove, and wherein the capillary sample collector is positioned within the groove.

18. The device of claim 17, wherein a gap is positioned between the open proximal end of the capillary sample collector and an end of the groove, wherein the gap has a length, and wherein the length of the gap is less than an external diameter of the capillary sample collector.

19. The device of claim 17, wherein a casing wall at the end of the groove forms an angle with the test strip sample pad, and wherein the angle is less than 90°.

20. The device of claim 19, wherein the angle is less than 60°.

21. The device of claim 1, further comprising a gasket positioned between the casing first part and the casing second part, wherein the capillary sample collector extends through the gasket.

22. The device of claim 1, wherein the device is configured to collect a fluid sample having a volume that is between 0.5 microliters and 100 microliters.

23. The device of claim 1, wherein the test strip further comprises a conjugate pad positioned adjacent the sample pad, wherein a proximal end of the sample pad is positioned above a distal end of the conjugate pad such that the sample pad has a bend angle, and wherein the bend angle is less than 30°.

24. The device of claim 1, wherein fluid collected in the capillary sample tube is visible from outside the device.

25. The device of claim 1, wherein diagnostic lines of the test strip are visible from outside the device.

26. The device of claim 1, wherein the casing first part and the casing second part are joined to one another to form a cylindrical casing.

27. The device of claim 26, wherein the cylindrical casing is shaped to form an airtight seal when the device is inserted into a tube housing a liquid reagent.

28. A method, comprising:
providing a capillary sample collector having a distal end and a proximal end, wherein the proximal end of the capillary sample collector has a dispensing opening;
collecting a fluid sample at the distal end of the capillary sample collector, wherein the capillary sample collector includes a hydrophilic portion, and wherein the capillary sample collector is configured to limit an amount of the fluid sample;
driving a reagent fluid into the distal end of the capillary sample collector, whereby the reagent fluid drives the fluid sample toward the dispensing opening; and
dispensing the fluid sample and at least a portion of the reagent fluid through the dispensing opening and onto a test strip sample pad,
wherein a flow rate of the sample fluid within the test strip sample pad in a direction toward a conjugate pad is greater than a flow rate of the sample fluid within the test strip sample pad in a direction away from the conjugate pad.

29. The method of claim 28, wherein the capillary sample collector comprises a burst valve between the proximal end and the distal end, and wherein the burst valve is configured to limit the amount of the fluid sample.

30. The method of claim 28, wherein the capillary sample collector further comprises a hydrophobic portion positioned between the hydrophilic portion and the proximal end, and wherein a size of the hydrophilic portion is configured to limit the amount of the fluid sample.

31. The method of claim 28, wherein a dilution of the fluid sample by the reagent fluid is less than 10%.

32. The method of claim 28, wherein the fluid sample is dispensed onto the sample pad at a tilt angle of less than 10°.

33. The method of claim 28, wherein a flow velocity of the fluid sample along the sample pad is between 0.1 mm/s and 1 mm/s.
